SEQUENCE LISTING (4 sequences)

SEQ ID NO: 1 - protein, 294 aa, Artificial
  a wild type VDAC2 polypeptide
MATHGQTCARPMCIPPSYADLGKXARDIFNKGFGFGLVKLDVKTKSCSGVEFSTSGSSNTDTGKVTGTLETKYKWCEYGLTFTEKWNTDNTLGTEIAIEDQICQGLKLTFDTTFSPNTGKKSGKIKSSYKRECINLGCDVDFDFAGPAIHGSAVFGYEGWLAGYQMTFDSAKSKLTRNNFAVGYRTGDFQLHTNVNDGTEFGGSIYQKVCEDLDTSVNLAWTSGTNCTRFGIAAKYQLDPTASISAKVNNSSLIGVGYTQTLRPGVKLTLSALVDGKSINAGGHKVGLALELEA

SEQ ID NO: 2 - protein, 295 aa, Artificial
  a wild type VDAC2 polypeptide
MAECCVPVCPRPMCIPPPYADLGKAARDIFNKGFGFGLVKLDVKTKSCSGVEFSTSGSSNTDTGKVSGTLETKYKWCEYGLTFTEKWNTDNTLGTEIAIEDQICQGLKLTFDTTFSPNTGKKSGKIKSAYKRECINLGCDVDFDFAGPAIHGSAVFGYEGWLAGYQMTFDSAKSKLTRSNFAVGYRTGDFQLHTNVNNGTEFGGSIYQKVCEDFDTSVNLAWTSGTNCTRFGIAAKYQLDPTASISAKVNNSSLIGVGYTQTLRPGVKLTLSALVDGKSFNAGGHKLGLALELEA

SEQ ID NO: 3 - protein, 10 aa, Artificial
  partial VDAC2 protein sequence
KVSGTLETKY

SEQ ID NO: 4 - protein, 14 aa, Artificial
  partial VDAC2 protein sequence
KYQLDPTASISAKV

(19) United States
(12) Patent Application Publication
(10) Pub. No.: US 2005/0085420 A1
(43) Pub. Date: Apr. 21, 2005

(54) Method of modulating apoptosis and compositions thereof

(76) Inventors: Stanley Korsmeyer, Weston, MA (US); Emily Cheng, Ladue, MO (US)

Correspondence Address: MINTZ, LEVIN, COHN, FERRIS, GLOVSKY;AND POPEO, P.C., ONE FINANCIAL CENTER, BOSTON, MA, 02111, US

(21) Appl. No.: 10/897,485

(22) Filed: Jul. 22, 2004

Related U.S. Application Data
(60) Provisional application No. 60/489,556, filed on Jul. 22, 2003.

(51) Int. Cl. A61K 38/17

(52) U.S. Cl. 514/12

(57) ABSTRACT

Disclosed herein are methods for modulating apoptosis. Methods are provided for inhibiting or preventing apoptosis by promoting formation of a complex of BAK and Voltage-Dependent Anion Channel 2 (VDAC2). The invention also provides methods of promoting or inducing apoptosis by disrupting or inhibiting formation of a VDAC2/BAK complex. Also disclosed are methods of screening for compounds that promote or disrupt the VDAC2/BAK complex.

### RELATED APPLICATIONS

This application claims priority to provisional application U.S. Ser. No. 60/489,556, filed Jul. 22, 2003, which is herein incorporated by reference in its entirety.

### STATEMENT AS TO FEDERALLY SPONSORED RESEARCH

This invention was made with U.S. government support under NIH grant R37CA50239.

### FIELD OF THE INVENTION

This invention relates generally to methods and compositions for the regulation of apoptosis.

### BACKGROUND OF THE INVENTION

Programmed cell death, referred to as apoptosis, plays an indispensable role in the development and maintenance of tissue homeostasis within all multicellular organisms. Genetic and molecular analysis from nematodes to humans has indicated that the apoptotic pathway of cellular suicide is highly conserved. In addition to being essential for normal development and maintenance, apoptosis is important in the defense against viral infection and in preventing the emergence of cancer.

Diverse intrinsic death signals emanating from multiple subcellular locales all induce the release of cytochrome c from mitochondria to activate Apaf-1 and result in effector caspase activation. The BCL-2 family of pro- and anti-apoptotic proteins constitute a critical control point for apoptosis. Proteins in the BCL-2 family are major regulators of the commitment to programmed cell death as well as executioners of death signals at the mitochondrion. Members of this family include both pro- and anti-apoptotic proteins and share homology in up to four conserved regions termed BCL-2 homology (BH) 1-4 domains. The family can be divided into three main sub-classes: anti-apoptotic proteins, pro-apoptotic proteins, “BH3-only” proteins.

The anti-apoptotic proteins, which include BCL-2 and BCL-X_{L}, are all “multidomain,” sharing homology throughout all four BH domains. However, the pro-apoptotic proteins can be further subdivided and include multidomain proteins, such as BAX and BAK, which possess sequence homology in BH1-3 domains. The more distantly related “BH3-only” proteins are to date all pro-apoptotic and share sequence homology within the amphipathic α-helical BH3 region, which is required for their apoptotic function.

The “BH3-only molecules” constitute the third subset of the BCL-2 family and include, for example, BID, NOXA, PUMA, BIK, BIM and BAD. These proteins share sequence homology only in the amphipathic α-helical BH3 region which mutation analysis indicated is required in pro-apoptotic members for their death activity. Moreover, the BH3-only proteins require this domain to demonstrate binding to “multidomain” BCL-2 family members. Multiple binding assays, including yeast two-hybrid, co-immunoprecipitation from detergent solubilized cell lysates and in-vitro pull down experiments indicate that individual BH3-only molecules display some selectivity for multidomain BCL-2 members. The BID protein binds pro-apoptotic BAX and BAK as well as anti-apoptotic BCL-2 and BCL-X_{L}. In contrast, BAD, NOXA and BIM as intact molecules display preferential binding to anti-apoptotic members.

The upstream BH3-only family members respond to select death signals and subsequently trigger the activation of the “multidomain” death effectors BAX and BAK. BAX and BAK constitute an essential gateway to the intrinsic death pathway operating both at the level of mitochondria and endoplasmic reticulum Ca²⁺ dynamics.

In viable cells, the BAK and BAX proteins exist as monomers. In response to a variety of death stimuli, however, inactive BAX, which is located in the cytosol or loosely attached to membranes, inserts deeply into the outer mitochondrial membrane as a homo-oligomerized multimer. Inactive BAK resides at the mitochondrion where it also undergoes an allosteric conformational change in response to death signals, which includes homo-oligomerization. Cells deficient in both BAX and BAK are resistant to a wide variety of death stimuli that emanate from multiple locations within the cell.

Activated, homo-oligomerized BAX or BAK results in the permeabilization of the outer mitochondrial membrane (OMM) and the release of proteins including cytochrome c, which initiates a caspase cascade and contributes to organelle dysfunction. Conversely, cells protected by adequate levels of anti-apoptotic BCL-2 or BCL-X_{L} bind and sequester translocated BH3-only molecules in stable complexes, preventing activation of BAX and BAK.

However, the mechanisms by which viable cells keep the potentially lethal pro-apoptotic BAX and BAK molecules in check are incompletely understood. Accordingly, there exists a need for methods and compositions that modulate apoptosis mediated by BAK or BAX.

### SUMMARY OF THE INVENTION

The present invention is based on the discovery that VDAC2, a VDAC isoform present in mammals, interacts with and inhibits BAK activation.

The invention provides methods for preventing apoptosis in a cell by contacting the cell with an anti-apoptotic compound. The anti-apoptotic compound prevents the homo-oligomerization of BAK and the induction of apoptosis. An anti-apoptotic compound includes, for example, wild-type VDAC2 proteins, VDAC2/BAK stabilizer compounds and VDAC2 mimetics. A VDAC2/BAK stabilizer compound is a compound that modulates the interaction between a VDAC2 protein and a BAK protein. By “modulating the interaction between VDAC2 and BAK” is meant that the VDAC2/BAK stabilizer compound promotes the formation of a VDAC2/BAK complex. By promoting the formation of the VDAC2/BAK complex, VDAC2 is prevented from dissociating from BAK, thereby preventing the homo-oligomerization of BAK and preventing the induction of apoptosis. The VDAC2/BAK stabilizer compound interacts with VDAC2, BAK or both, to prevent the homo-oligomerization of BAK. A VDAC2/BAK stabilizer compound is, for example, a small molecule, a protein, a peptide or a peptide fragment. A VDAC2 mimetic is a compound that interacts with an endogenous BAK protein, and prevents the homo-oligomerization of BAK and prevents the induction of apoptosis. A VDAC2 mimetic is, for example, a mutated VDAC2 peptide or a small molecule that mimics the biological function of endogenous VDAC2 . A VDAC2/BAK stabilizer compound is, for example, a BH3 mutein that does not cause the dissociation of the VDAC2/BAK complex.

The anti-apoptotic compounds of the invention are useful in preventing or alleviating an apoptosis-associated disorder or a symptom of an apoptosis-associated disorder. Apoptosis-associated disorders and/or a symptom of an apoptosis-associated disorder are prevented or alleviated by administering an anti-apoptotic compound to a subject. An apoptosis-associated disorder is, for example, stroke, myocardial infarction, hypertension, septic shock, acquired immune deficiency syndrome (AIDS), organ transplantation or a neurodegenerative disorder such as Parkinson's disease, amytrophic lateral sclerosis (ALS), Alzheimer's disease, Huntington's disease or immune deficiency.

In another aspect, the invention provides methods for promoting apoptosis in a cell by contacting the cell with a VDAC2/BAK inhibitor compound, such that the VDAC2/BAK inhibitor compound prevents a VDAC2 protein and a BAK protein from interacting and forming a VDAC2/BAK complex. Alternatively, the VDAC2/BAK inhibitor compound promotes dissociation of the VDAC2/BAK complex. Therefore, the BAK protein is free to homo-oligomerize, thereby promoting apoptosis. The VDAC2/BAK inhibitor compound is, for example, a BID protein, including tBID, the activated form of BID, a PUMA protein, a BIM protein, a BAD protein, a NOXA protein, a BH3 domain peptide, e.g., a BH3-only molecule, a BID mutein, a BIM mutein, a BAD mutein, a NOXA mutein, another BH3 domain-only mutein, a VDAC2 mute in, a BAK mutein or combinations thereof. Alternatively, the VDAC2/BAK inhibitor compound is an anti-VDAC2 antibody. VDAC2/BAK inhibitor compounds include, for example, a BH3 domain peptide, a BH3 only peptide mimetic or a BH3 mutein that interacts with a VDAC2 protein while the VDAC2 protein is associated with BAK in a VDAC2/BAK complex, thereby releasing BAK from the VDAC2/BAK complex.

The VDAC2/BAK inhibitor compounds of the invention are useful in preventing or alleviating a cell-proliferative disorder or a symptom of a cell-proliferative disorder. Cell-proliferative disorders and/or a symptom of a cell-proliferative disorder are prevented or alleviated by administering a VDAC2/BAK inhibitor compound to a subject. The VDAC2/BAK inhibitor compound modulates interaction between a VDAC2 protein and a BAK protein to prevent a VDAC2/BAK complex. The cell-proliferative disorder is, for example, neoplasias including solid tumor cancers (e.g., lung, pancreas, stomach, colon, rectum, kidney, breast, cervical/uterine, ovarian, testicular, melanoma, head and neck, and esophageal cancers), as well as hematogenous cancers, such as leukemias and lymphomas, non-malignant neoplasias, cellular expansions due to DNA viruses such as Epstein-Barr virus, African swine fever virus and adenovirus, lymphoproliferative conditions, arthritis, inflammation, or autoimmune diseases.

The present invention includes methods for screening for an anti-apoptotic compound, such as, for example, a VDAC2/BAK stabilizer compound or a VDAC2 mimetic. Anti-apoptotic compounds are identified using any screening methods known to those skilled in the art, such as, for example, high throughput screening of various compound libraries, yeast two-hybrid screening, reverse yeast two-hybrid screening, and yeast three-hybrid screening. A cell-based screening assay is used to identify anti-apoptotic compounds according to the invention. In these methods, a cell population is contacted with a candidate compound, and the level of apoptosis of the cell population is determined. The cell population contains at least one cell having a VDAC2 protein and a BAK protein. A decrease in the level of apoptosis of the cell population in the presence of a candidate anti-apoptotic compound, when compared to the normal, control level of apoptosis for that cell population in the absence of the candidate compound, indicates that the candidate compound is an anti-apoptotic compound.

The invention also includes methods for screening for a VDAC2/BAK inhibitor compound. Anti-apoptotic compounds are identified using any screening methods known to those skilled in the art, such as, for example, high throughput screening of various compound libraries, yeast two-hybrid screening, reverse yeast two-hybrid screening, and yeast three-hybrid screening. A cell-based screening assay is used to identify anti-apoptotic compounds according to the invention. According to these methods, a cell population is contacted with a candidate compound, and the level of apoptosis of the cell population is determined. The cell population contains at least one cell having a VDAC2 protein and a BAK protein. An increase in the level of apoptosis for that cell population in the presence of the candidate VDAC2/BAK inhibitor compound, when compared to the normal, control level of apoptosis in the absence of the candidate compound, indicates that the candidate compound is a VDAC2/BAK inhibitor compound.

The normal, control level of apoptosis is the level of apoptosis of a given cell, or population of cells, under standard cell culture techniques known in the art. The cell population is contacted in vivo or in vitro.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and are not intended to be limiting.

Other features and advantages of the invention will be apparent from the following detailed description and claims.

### BRIEF DESCRIPTION OF THE DRAWINGS

FIG. 1 are photographs of immunoblots developed with an anti-BAK antibody. The immunoblots depict characterization of protein “X” that interacts with BAK.

FIG. 1A is an illustration depicting isolation of mitochondria (50 μg) from wild-type (wt) mouse embryonic fibroblasts (MEFs). The isolated mitochondria were incubated with recombinant tBID protein (50 ng) at 30° C. for 30 min, followed by treatment with the cross-linkers disuccinimidyl suberate (DSS) (10 mM) or bismaleimidohexane (BMH) (10 mM) at room temperature for 30 minutes. Higher order BAK complexes were identified by immunoblot developed with an anti-BAK antibody. The asterisk denotes an intrachain cross-link of the inactive BAK conformer.

FIG. 1B is an illustration depicting the treatment of FL5.12 cells with either TNFα plus cyclohexamide (CHX) for 5 hours (lane 3), etoposide (VP16) for 24 hours (lane 4), or deprived of IL-3 for 18 hours (lane 5), followed by incubation with 10 mM DSS. The BAK-X complex was detected in whole cell lysates by an immunoblot developed with anti-BAK antibody.

FIG. 1C is an illustration depicting treatment of mitochondria (50 μg) isolated from mouse liver, Bal17, Jurkat, and FL5.12 cells with 50 ng tBID protein at 30° C. for 30 min, followed by DSS cross-linking. The BAK-X complex was detected by an immunoblot developed with anti-BAK antibody.

FIG. 1D is a photograph depicting isolated mitochondria from wt MEFs, in which the isolated mitochondria were treated with DSS 24 hours following retroviral transduction of tBID, BIM, or BAD. The BAK-X complex was detected by an immunoblot developed with an anti-BAK antibody.

FIG. 1E is a photograph depicting isolated liver mitochondria (50 μg) which were incubated with 5 μl of in vitro transcribed-translated wt or mutant tBID at 30° C. for 30 min, followed by DSS cross-linking. The BAK-X complex was detected by an immunoblot developed with anti-BAK antibody. The asterisk denotes a cross-reactive protein.

FIG. 1F is a photograph depicting Bax, Bak doubly-deficient MEFs reconstituted with wt or mutant Bak (mBH3 or mBH1), wherein the Bax, Bak doubly-deficient MEFs were treated with DSS. The BAK-X complex was detected in whole cell lysates by an immunoblot developed with anti-BAK antibody.

FIG. 2 are illustrations and a schematic diagram that depict the identification of VDAC2 as the BAK-interacting partner that regulates BAK conformation.

FIG. 2A is schematic representation of the purification scheme for Flag-tagged BAK-X complex.

FIG. 2B is a photograph depicting aliquots of the Flag-peptide eluent from anti-Flag column which were analyzed by silver stain (lane 1) and immunoblot developed with anti-BAK antibody (lane 2). The silver-stained band corresponding to Flag-BAK-X was subjected to tryptic digestion and Liquid chromatography, tandem mass spectrometry. Asterisks indicate the peptide sequences of VDAC2 revealed by mass spectrometry.

FIG. 2C is a photograph depicting cellular extracts (20 μg protein) from wt, vdac1 -/-, vdac2 -/-, or vdac2 reconstituted vdac2 -/- ES cells, which were treated with 10 mM DSS and were analyzed by immunoblot developed with an anti-BAK antibody. Reconstitution of vdac2 into vdac2 -/- cells was achieved by retroviral transduction.

FIG. 2D is a photograph depicting mitochondria (50 μg) isolated from wt, vdac1 -/- (v1⁻), or vdac2 -/- (v2⁻) ES cells that were treated with 30 μg/ml trypsin. The trypsin sensitivity pattern of BAK was assessed by immunoblot using two distinct epitope-specific anti-BAK antibodies (upper panel, anti-BAK NT; lower panel, anti-BAK G23).

FIG. 2E is a photograph depicting mitochondria isolated from wt MEFs (upper panel) or Bax, Bak doubly deficient MEFs (lower panel), which were transduced with retrovirus expressing N-terminal HA-tagged VDAC2, BAK or BAX and were solubilized in either 1% CHAPS or 0.2% NP-40 buffer, followed by immunoprecipitation with an anti-HA antibody. The immunoprecipitates were analyzed by an immunoblot developed with anti-HA-Biotin, anti-BAK, anti-BAX, or anti-VDAC2 Abs as indicated.

FIG. 3 are graphs and histograms that depict regulation of BAK-dependent apoptosis by VDAC2.

FIG. 3A are two graphs illustrating Bax -/- or Bak -/- MEFs at 2 days following retroviral expression of VDAC1, VDAC2, or GFP (MIG vector control), wherein the Bax -/- or Bak -/- MEFs were treated with 1.2 μM staurosporine (STS). Cell death was quantitated by flow cytometric detection of Annexin-V staining at indicated time points. Values shown are mean ±1 SD of three independent experiments.

FIG. 3B is a histogram depicting Bax -/- or Bak -/- MEFs at 2 days following retroviral expression of VDAC2 or GFP (MIG vector control), wherein the Bax -/- or Bak -/- MEFs were infected with tBID-expressing retrovirus. Cell death was quantitated by flow cytometric detection of Annexin-V staining at 24 hr. Values shown are mean ±1 SD of three independent experiments.

FIG. 3C is a histogram depicting wild-type, vdac1 -/-, or vdac2 -/- ES cells that were treated with staurosporine (1.2 μM) for 6 hours or etoposide (10 μg/ml) for 12 hours. Cell death was quantitated by flow cytometric detection of Annexin-V staining. Values shown are mean ±1 SD of three independent experiments.

FIG. 3D is a graph illustrating treatment of primary wt or vdac2 -/- MEFs with 1.2 μM staurosporine. Cell death was quantitated by flow cytometric detection of Annexin-V staining at indicated time points. Values shown are mean ±1 SD of three independent experiments.

FIG. 3E is a histogram depicting infection of primary vdac2 -/- MEFs with retrovirus expressing GFP (MIG vector control) or VDAC2-IRES-GFP. At 36 hours the GFP-positive cells isolated by MoFlo sorting were treated with 1.2 μM staurosporine for 6 hr. Cell death was quantitated by flow cytometric detection of Annexin-V staining. Values shown are mean ±1 SD of three independent experiments.

FIG. 4 are graphs, photographs and histograms depicting comparison of apoptotic hallmarks in vdac2 -/- and wt cells.

FIG. 4A is a graph illustrating SV40-transformed wt or vdac2 -/- MEFs that were treated with 1 μM staurosporine (STS). Cell death was quantitated by flow cytometric detection of Annexin-V staining at indicated time points. Values shown are mean of two independent experiments. The same increased susceptibility to apoptosis was noted in the vdac2 null cell line as in primary MEFs.

FIG. 4B is a graph illustrating the mitochondrial membrane potential (Δψₘ) of wt or vdac2 -/- MEF cell lines following treatment with 1 μM STS. The mitochondrial membrane potential (Δψₘ) was assessed by flow cytometric detection of TMRE uptake at indicated time points. Values shown are mean of two independent experiments.

FIG. 4C is a photograph depicting treatment of wt or vdac2 -/- MEF cell lines with 1 μM STS. The wt or vdac2 -/- MEF cell lines were subcellularly fractionated (see method) at indicated time points. Cytochrome c distribution in the cytosol versus mitochondria was assessed by immunoblot developed with an anti-cytochrome c antibody.

FIG. 4D is a photograph depicting two-color fluorescence microscopy of wt and vdac2 -/- MEF lines at 4 hours following 1 μM STS. Red indicates cytochrome c and blue is Hoechst staining of DNA. Analysis of multiple fields indicates that 83% of vdac2 -/- cells, but only 16% of wt cells display diffuse cytosolic staining for cytochrome c at 4 hours post STS.

FIG. 4E is histogram depicting cell lysates from wt or vdac2 -/- MEF lines treated with 1 μM STS for 5 hours or TNFα (2 ng/ml) plus cyclohexamide (2 μg/ml) for 12 hours were assessed for effector caspase activity by a DEVD-AFC cleavage assay. Values shown are mean ±1 SD of three independent experiments.

FIG. 4F is a photograph depicting mitochondria isolated from wt or vdac2 -/- MEF cell lines with or without treatment with STS for 5 hours were solubilized in 2% CHAPS buffer. 200 μg of lysate was subjected to Superdex 200 (HR 10/30) gel filtration column chromatography. Fractions were analyzed by an immunoblot developed with anti-BAK antibody.

FIG. 5 is a photograph illustrating that BCL-2 does not substantially interact with BAK. Mitochondria (100 μg) isolated from FL5.12 cells expressing BCL-2 were untreated or treated with recombinant tBID protein at 30° C. for 30 min, and then lysed in 1% CHAPS buffer (1% CHAPS, 142.5 mM KCl, 5 mM MgCl₂, 1 mM EDTA, 1 mM EGTA, 20 mM Tris, pH7.5). The lysates were immunoprecipitated with an anti-BCL-2 antibody. The immunoprecipitates were analyzed by immunoblot developed with anti-BAK and anti-BCL-2 antibody.

FIG. 6 are two graphs that depict Bax -/- or Bak -/- MEFs at 2 days following retroviral expression of VDAC1, VDAC2, or GFP (MIG vector control), wherein the Bax -/- or Bak -/- MEFs were treated with 10 μg/ml etoposide. Cell death was quantitated by flow cytometric detection of Annexin-V staining at indicated time points. Values shown are mean ±1 SD of three independent experiments.

FIG. 7A is a graph that illustrates primary wt or vdac2 -/- MEFs that were treated with 100 μM etoposide. Cell death was quantitated by flow cytometric detection of Annexin-V staining at indicated time points. Values shown are mean ±1 SD of three independent experiments.

FIG. 7B is a graph that depicts primary wt, vdac1 -/- or vdac3 -/- MEFs that were treated with 1.2 μM staurosporine. Cell death as quantitated by flow cytometric detection of Annexin-V was similar in all cells.

FIG. 8 is a graph that illustrates the time course of staurosporine-induced caspase activation. Lysates from wt or vdac2 -/- MEF lines treated with 1 μM staurosporine at indicated time points were assessed for DEVD-AFC cleavage activity. Data presented are the fold activation of DEVD-AFC cleavage activity compared to the basal activity at 0 hour.

FIG. 9 is a photograph depicting that a hemagglutinin (HA)-tagged version of VDAC2, when immunoprecipitated from fibroblasts lacking the vdac1 and vdac3 isoforms, is associated with the BH3-only molecule p15 tBID, and that the mutant BH3 domain of tBID, m l 11.4, does not interact with VDAC2.

FIG. 10A is a photograph depicting that an HA-tagged version of VDAC2, when immunoprecipitated in either CHAPS or NP40 detergent, is associated with the BH3-only molecule BIM-EL.

FIG. 10B is a photograph depicting the association between the BH3-only molecule BIM-EL and HA-BAX, HA-BAK or HA-VDAC2 reconstituted in Bax -/-, Bak -/- double knock out (DKO) mouse embryonic fibroblasts (MEFs).

FIG. 11A is a photograph depicting that the BH3-only molecule PUMA is associated with VDAC2 in cells lacking the other VDAC isoforms, VDAC1 and VDAC3.

FIG. 11B is a photograph depicting that, in death resistant Bax -/-, Bak -/- DKO cells, the amount of PUMA increases following a DNA damaging death signal of Etoposide. The induced PUMA is seen to associate with an HA-tagged version of VDAC2.

### DETAILED DESCRIPTION OF THE INVENTION

The present invention provides compositions and methods for modulating apoptosis. The invention is based, in part, on the discovery of a novel interaction between Voltage-Dependent Anion Channel-2 (VDAC2) and BAK. BAK is a “multidomain” pro-apoptotic molecule that is required to initiate the mitochondrial pathway of apoptosis. Other multidomain pro-apoptotic molecules include BAX. When monomers, these multidomain pro-apoptotic molecules are inactive, however, upon exposure to cell death stimuli, BAK and BAX become activated and form homo-oligomers. Activated, homo-oligomerized BAX or BAK results in the permeabilization of the outer mitochondrial membrane (OMM) and the release of proteins including cytochrome c, which initiates a caspase cascade and contributes to organelle dysfunction. Prior to exposure to death stimuli, BAX and BAK monomers do not interact appreciably with anti-apoptotic BCL-2 family members. In viable cells, BAK is an integral mitochondrial membrane protein in cells not exposed to death stimuli, however, upon activation by BH3-only family members, BAK has been found to interact with anti-apoptotic BCL-2 family members (FIG. 5).

In viable cells, BAK was found complexed with VDAC2, a VDAC isoform found only in mammals and present in low abundance. VDAC2 was found to interact with the inactive, monomer conformation of BAK. Cells that were deficient for VDAC2 exhibited enhanced BAK oligomerization and were more susceptible to apoptotic death. In contrast, cells that were lacking the more abundant VDAC1 isoform did not exhibit such characteristics. Overexpression of VDAC2 selectively prevented BAK activation and inhibited the mitochondrial apoptotic pathway. As shown in the Examples provided herein, the “BH3-only” molecules, such as BID, BIM, BAD or NOXA, were found to displace VDAC2 from BAK, enabling homo-oligomerization of BAK and triggering apoptosis. Thus, VDAC2 regulates the activity (e.g., apoptosis) of BAK.

The present invention is directed to methods for promoting or inhibiting apoptosis and methods for treating or preventing apoptosis-associated disorders or cell-proliferative disorders by promoting or disrupting protein complexes of BAK and a protein that interacts with (ie., binds to) BAK. Specifically, these methods are directed to promoting or disrupting complexes of BAK, and derivatives, fragments and analogs of BAK, with VDAC2, and its derivatives, fragments and analogs (a complex formed between a BAK protein and a VDAC2 protein is designated herein as a “VDAC2/BAK complex”). The present invention is further directed to methods of screening for compounds that promote or disrupt the VDAC2/BAK complex.

Methods of Inhibiting Apoptosis

The present invention provides methods of inhibiting apoptosis. Apoptosis, also known as programmed cell death, plays a role in development, aging and in various pathologic conditions. In developing organisms, both vertebrate and invertebrate, cells die in particular positions at particular times as part of the normal morphogenetic process. The process of apoptosis is characterized by, but not limited to, several events. Cells lose their cell junctions and microvilli, the cytoplasm condenses and nuclear chromatin marginates into a number of discrete masses. As the nucleus fragments, the cytoplasm contracts and mitochondria and ribosomes become densely compacted. After dilation of the endoplasmic reticulum and its fusion with the plasma membrane, the cell breaks up into several membrane-bound vesicles, apoptotic bodies, which are usually phagocytosed by adjacent bodies. As fragmentation of chromatin into oligonucleotides oligonucleosomal fragments is characteristic of the final stages of apoptosis, DNA cleavage patterns can be used as and in vitro assay for its occurrence. (Cory, Nature 367: 317-18, 1994).

Inhibition of apoptosis is useful in the treatment of various disorders associated with aberrant cell death. For example, it is desirable to inhibit apoptosis in certain disease conditions such as in the treatment of immunodeficiency diseases, including AIDS; senescence; neurodegenerative diseases such as Alzheimer's Disease, Huntington's Disease, Parkinson's Disease or Amyotrophic Lateral Sclerosis (ALS); ischemia and reperfusion; infertility; wound-healing; stroke; myocardial infarction; hypertension; septic shock; and organ transplantation.

Apoptosis is prevented in a cell by contacting the cell with an anti-apoptotic compound. An anti-apoptotic compound is a compound that prevents the homo-oligomerization of BAK and therefore the induction of apoptosis. For example, the anti-apoptotic compound interacts with BAK such that homo-oligomerization is decreased and/or prevented. An anti-apoptotic compound is for example, a wild-type VDAC2 protein, a VDAC2/BAK stabilizer compound or a VDAC2 mimetic.

VDAC2/BAK Stabilizer Compounds

As used herein, the term “VDAC2/BAK stabilizer compound” refers to a compound that promotes the formation of a VDAC2/BAK complex. By promoting the formation of the VDAC2/BAK complex, VDAC2 is prevented from dissociating from BAK, which prevents the homo-oligomerization of BAK and therefore prevents the induction of apoptosis. For example, the VDAC2/BAK stabilizer compound promotes the formation of the VDAC2/BAK complex by increasing the binding affinity of VDAC2 and BAK. By “increasing the binding affinity” is meant compared to the normal, control binding affinity of VDAC2 and BAK, such that VDAC2 is not displaced, and consequently, the VDAC2/BAK complex is not disrupted, in the presence of a death signal peptide, such as, for example, a BH3 only peptide (e.g., BID, BIM, BAD and/or NOXA).

VDAC2/BAK stabilizer compounds of the invention include, for example, an antibody, an antibody fragment, a peptide or a peptidomimetic that specifically binds a death signal peptide that interacts with BAK, thereby preventing the BH3 death signal peptide from disrupting the VDAC/BAK interaction and triggering apoptosis. Alternatively, VDAC2/BAK stabilizer compounds include a compound (e.g. an antibody, antibody fragment, peptide or peptidomimetic) that acts as a “stabilizing clamp” to prevent dissociation of the VDAC2/BAK complex, which prevents the homo-oligomerization of BAK and the induction of apoptosis. These stabilization clamp compounds bind to VDAC2, BAK or both, to prevent disruption of the VDAC2/BAK interaction.

VDAC2 Mimetic Compounds

As used herein, the term “VDAC2 mimetic” refers to a compound that mimics the biological function of endogenous VDAC2. The VDAC2 mimetic interacts with a BAK protein to form a VDAC2 mimetic/BAK complex, which prevents the homo-oligomerization of BAK and therefore preventing the induction of apoptosis. Biological function of endogenous VDAC2 includes, for example, binding to BAK. The VDAC2 mimetics inhibit BAK activation and act as substitutes for endogenous VDAC2 protein. Suitable VDAC2 mimetics include, for example, a mutated VDAC2 peptide, also referred to herein as an anti-apoptotic VDAC2 mutein or an anti-apoptotic VDAC2 mutein polypeptide. An anti-apoptotic VDAC2 mutein has an amino acid sequence that includes at least a fragment of a wild type (wt) VDAC2 protein, where one or more amino acid residues of the wt VDAC2 protein is mutated (e.g. substitution, insertion or deletion). As used herein, “wild type (wt) VDAC2 ” includes any wild-type VDAC2, whether native or recombinant, having the naturally occurring amino acid sequence of the full-length, native VDAC2, as shown in, e.g., U.S. Pat. No. 5,780,235; GenBank Accession Nos: NP_{—}003366; NP_{—}776911; NP_{—}112644; NP_{—}035825; P45880; B44422; Q9TT14; Q60930; P82013; P81155; CAB949711; BAB13474; AAF78964; AAF80116; AF80102; AAF73513; AAD40241; XP293180; NP_{—}035825 or Q60930. An anti-apoptotic VDAC2 mutein, is capable of forming a complex with the BAK polypeptide, such that the BAKIVDAC2 mutein complex is not disrupted in the presence of a death stimulus, such as, for example, BH3 only molecules ( e.g., BID, BIM, BAD or NOXA). For example, the anti-apoptotic VDAC2 mutein has a higher affinity for BAK than wt VDAC2. Optionally, the anti-apoptotic VDAC2 mutein binds BAK, but does not bind a death signal peptide, such as, for example, BID, BIM, BAD and NOXA. Other suitable VDAC2 mimetics include, for example, small molecules that mimic the biological function of endogenous VDAC2 and interact with a BAK protein. A “small molecule,” as used herein, refers, for example, to a protein or peptide that has a molecular weight of less than about 5 kD and preferably less than about 4 kD, or to an inorganic or organic compound having a molecular weigh of less than about 600 Daltons. Small molecules are, e.g., nucleic acids, peptides, polypeptides, peptidomimetics, carbohydrates, lipids or other organic or inorganic molecules.

The anti-apoptotic compound is added in an amount that is sufficient to enhance or promote binding between VDAC2 and BAK, to inhibit apoptosis. For example, an exogenous VDAC2 protein or a VDAC2 mimetic is introduced to a cell population such that it is expressed at a concentration that is higher than the normal cellular concentration of endogenous VDAC2. Higher than normal concentration are at least 2×, 5×, 10×, or greater, higher than the normal cellular concentration of endogenous VDAC2. The cell population that is exposed to, i.e., contacted with, the anti-apoptotic stabilizer compound is any number of cells, i.e., one or more cells, and is provided in vitro, in vivo, or ex vivo. Cells in which apoptosis is inhibited include, for example, stromal, epithelial, organ specific, or blood-derived cells. For example, the cells are differentiated fibroblasts and keratinocytes. Additionally, the cells are fibroblasts, keratinocytes (including outer root sheath cells), melanocytes, endothelial cells, pericytes, monocytes, lymphocytes (including plasma cells), thrombocytes, mast cells, adipocytes, muscle cells, hepatocytes, neurons, nerve or neuroglia cells, osteocytes, osteoblasts, corneal epithelial cells, chondrocytes, ectodermal cells, endodermal cells, mesodermal cells and/or adult or embryonic stem cells. Any cell in which inhibition of apoptosis is desirable may be used.

Apoptosis-associated disorders and symptoms thereof are treated, prevented or alleviated by administering to a subject in need thereof an anti-apoptotic compound. The anti-apoptotic compound is administered in an amount that is effective to modulate interaction between VDAC2 and BAK, such that apoptosis is inhibited. The subject is e.g., any mammal, e.g., a human, a primate, mouse, rat, dog, cat, cow, horse, pig.

To alleviate, prevent or treat a symptom of an apoptosis-associated disorder, an anti-apoptotic compound is administered in a therapeutically effective amount, such that the anti-apoptotic compound modulates interaction between VDAC2 and BAK, and the symptom is treated, prevented or alleviated. The term “therapeutically effective” means that the amount of anti-apoptotic compound, for example, which is used, is of sufficient quantity to ameliorate at least one symptom of an apoptosis-associated disorder. Symptoms of an apoptosis-associated disorder include, for example, memory loss, uncontrolled movements (e.g., dyskinesia, chorea), disorientation or confusion, mood swings, elevated creatine kinase levels, tremors, impaired balance, impaired ability to initiate movement (akinesia), slowness of movement (bradykinesia), difficulty swallowing (dysphagia), loss or impaired sense of smell (anosmia), fever, sore throat, headache, muscle aches, joint pain, enlarged lymph nodes, abdominal cramps, diarrhea, nausea, vomiting, chest pain, shortness of breath, fatigue, abnormal blood pressure (e.g., elevated or low, especially when standing), lightheadedness, heart palpitations, and rapid heart rate.

The VDAC2/BAK complex formation is detected by determining the level of cellular survival and/or proliferation in the presence of an anti-apoptotic compound and comparing it to the level of cellular survival and/or proliferation in the absence of the anti-apoptotic compound, either with or without an accompanying death stimulus, such as, for example, a death signal peptide (e.g., BID, BIM, BAD or NOXA). A similar level or an increase in the level of cellular survival and/or proliferation in the presence of the anti-apoptotic compound indicates the presence of the VDAC2/BAK complex. The level of cellular survival and/or proliferation is compared to a normal, control level of cellular survival and/or proliferation for the cell type that is contacted with the anti-apoptotic compound. The normal, control level of cellular survival and/or proliferation is determined by preparing control cells, which have not been contacted with the anti-apoptotic compound, and test cells, which are contacted with the anti-apoptotic compound, and monitoring the levels of cellular survival and/or proliferation for each population of cells. Alternatively, the normal, control level of cellular survival and/or proliferation can be determined by monitoring the level of cellular survival and/or proliferation of a cell (or a population of cells) for a period of time (e.g., 1 hour, 24 hrs, 48 hrs), then contacting the same cell (or cell population) with an anti-apoptotic compound and monitoring the level of cellular survival and/or proliferation in the presence of the anti-apoptotic compound, either in the presence of absence of an accompanying death stimulus. Cell survival and/or proliferation is measured by methods know in art. Alternatively, the normal, control level of cellular survival and/or proliferation can also be determined using a reference library of known rates of cellular survival and/or proliferation for a given cell type. For example, this level of cell survival and/or proliferation can be contained in published research, journal articles and other scientific resources.

Apoptosis is measured by any of a variety of methods known in the art. For example, in one embodiment, apoptosis is measured by DNA ladder formation by gel electrophoresis and of morphologic examination by electron microscopy. Alternatively, apoptosis is measured by flow cytometry. Flow cytometry permits rapid and quantitative measurements on apoptotic cells. Many different flow cytometric methods for the assessment of apoptosis in cells have been described. (Darzynkiewicz et al. Cytometry 13: 795-808, 1992). These methods measure apoptotic changes in cells by staining with various DNA dyes (i.e. propidium iodide (PI), DAPI, Hoechst 33342), however, techniques using the terminal deoxynucleotidyl transferase (TUNNEL) or nick translation assays have also been developed (Gorczyca et al. Cancer Res 53: 1945-1951, 1993). Recently, rapid flow cytometric staining methods that use Annexin V for detection of phosphatidylserine exposure on the cell surface as a marker of apoptosis have become commercially available. The newest flow cytometric assays measure Caspase-3 activity, an early marker of cells undergoing apoptosis and kits for performing this assays are commercially available. (Nicholson et al. Nature 376: 37-43, 1995).

Methods of Promoting Apoptosis

Also included in the invention are methods of promoting or inducing apoptosis. Promotion or induction of apoptosis is useful in the treatment of various disorders associated with aberrant cell proliferation, such as, for example, neoplasias including solid tumor cancers (e.g., lung, pancreas, stomach, colon, rectum, kidney, breast, cervical/uterine, ovarian, testicular, melanoma, head and neck, and esophageal cancers), as well as hematogenous cancers, such as leukemias and lymphomas, and non-malignant neoplasias. Disease conditions, such as lymphoproliferative conditions, cancer including drug resistant cancer, arthritis, inflammation, autoimmune diseases and the like result from a defect in cell death regulation. Other disease conditions are affected by the development of a defective apoptotic response. For example, neoplasias may result, at least in part, from an apoptosis-resistant state in which cell proliferation signals inappropriately exceed cell death signals. Furthermore, DNA viruses such as Epstein-Barr virus, African swine fever virus and adenovirus, parasitize the host cellular machinery to drive their own replication and at the same time modulate apoptosis to repress cell death and allow the target cell to reproduce the virus. Accordingly, it would be desirable to promote or induce apoptotic mechanisms in such disease conditions.

Apoptosis is promoted or induced in a cell by contacting a cell with a VDAC2/BAK inhibitor compound. The term “a VDAC2/BAK inhibitor compound,” as used herein, refers to a compound that disrupt (ie., inhibit or prevent) interaction between VDAC2 and BAK. Disruption of the VDAC2/BAK complex results in BAK homo-oligomerization when activated by a death signal, including for example, peptides such as a BH3 only molecule (e.g., BID, BIM, BAD and/or NOXA). Disruption of the VDAC2/BAK complex includes, for example, a displacement reaction in which another protein, peptide or small molecule, e.g., a BH3 only protein or a BH3 only mimetic, competes for VDAC2, thereby displacing VDAC2 from the VDAC2/BAK complex and freeing BAK to homo-oligomerize.

The VDAC2/BAK inhibitor compound according to the present invention is, for example, a pro-apoptotic VDAC2 mutein that competes with the wild-type, endogenous VDAC2 for binding BAK, but does not prevent BAK activation. Alternatively, the VDAC2/BAK inhibitor compound is a BAK mutein that binds native, endogenous VDAC2 and prevent VDAC2 from binding to, and inhibiting, BAK. The VDAC2/BAK inhibitor compound is, for example, a BID protein, a BIM protein, a PUMA protein, a BAD protein, a NOXA protein, a BH3 domain peptide, a BID mutein, a BIM mutein, a BAD mutein, a NOXA mutein, a BH3 domain-only molecule mutein or peptide mimetic that prevents VDAC2 from interacting with (i.e., binding) BAK, but does not prevent BAK activation, or a BH3-only mutein or mimetic that promotes dissociation of the VDAC2/BAK complex. Preferably, the BH3 domain peptide is a BH3 only protein such as BID, tBID, BIM, BIM-EL or PUMA, which interacts with VDAC2 while VDAC2 is associated with BAK in the VDAC2/BAK complex, thereby releasing BAK from the VDAC2/BAK complex. Once uncomplexed from VDAC2, BAK homo-oligomerizes and initiates apoptosis. Other suitable VDAC2/BAK inhibitor compounds include an antibody or antibody fragment that immunospecifically binds to at least a portion of the VDAC2 protein that binds to the BAK protein, or an antibody or antibody fragment that immunospecifically binds to at least a portion of the BAK protein. Alternatively, the VDAC2/BAK inhibitor compound is a small molecule that disrupts VDAC2/BAK binding. Small molecule VDAC2/BAK inhibitor compounds are identified by screening a small molecule chemical library for compounds that disrupt the VDAC2/BAK complex.

The VDAC2/BAK inhibitor compound is added in an amount that is sufficient to disrupt or inhibit binding between VDAC2 and BAK, thereby promoting apoptosis. The cell population that is exposed to, i.e., contacted with, the VDAC2/BAK inhibitor compound is any number of cells, i.e., one or more cells, and is provided in vitro, in vivo, or ex vivo. Suitable cells for use with the methods of the present invention include, for example, stromal, epithelial, organ specific, or blood-derived cells. For example, the cells are differentiated fibroblasts and keratinocytes. Additionally, the cells are fibroblasts, keratinocytes (including outer root sheath cells), melanocytes, endothelial cells, pericytes, monocytes, lymphocytes (including plasma cells), thrombocytes, mast cells, adipocytes, muscle cells, hepatocytes, neurons, nerve or neuroglia cells, osteocytes, osteoblasts, corneal epithelial cells, chondrocytes, ectodermal cells, endodermal cells, mesodermal cells and/or adult or embryonic stem cells.

Cell-proliferative disorders, and symptoms of cell-proliferative disorders, are treated, prevented or alleviated by administering to a subject in need thereof a VDAC2/BAK inhibitor compound in an amount sufficient to disrupt or inhibit binding between VDAC2 and BAK, thereby promoting apoptosis. The subject is e.g., any mammal, e.g., a human, a primate, mouse, rat, dog, cat, cow, horse, pig. For example, the amount sufficient to disrupt or inhibit binding between VDAC2 and BAK is a therapeutically effective amount a VDAC2/BAK inhibitor compound. The term “therapeutically effective amount” refers to the amount of VDAC2/BAK inhibitor compound that is sufficient to treat, prevent or ameliorate a cell-proliferative disorder, or at least one symptom of a cell-proliferative disorder. Symptoms of a cell-proliferative disorder include, for example, severe headache, fever, swollen lymph nodes or glands, palpitations, rapid heart rate, chest pain, sweating, abdominal pain, weight loss, anxiety, diarrhea, cough with blood, fatigue, muscle fatigue, back pain, increased thirst, increased urination, nausea and mucous membrane inflammation.

The presence of a VDAC2/BAK inhibitor compound, and consequently, the inhibition of the VDAC2/BAK complex, is detected by determining the level of cellular survival and/or proliferation in the presence a VDAC2/BAK inhibitor compound of the present invention and comparing it to the level of cellular survival and/or proliferation in the absence of the VDAC2/BAK inhibitor compound, in the presence or absence of a death stimulus. A decrease in the level of cellular survival and/or proliferation indicates absence or disruption of the VDAC2/BAK complex. The normal, control level of cellular survival and/or proliferation can be determined using a variety of methods, such as those described above. Apoptosis, cellular survival and/or proliferation are measured using any of the methods known in the art, as described above.

VDAC2/BAK Muteins

VDAC2 mimetics and VDAC2/BAK inhibitor compounds of the invention include, for example, a mutated VDAC2 protein, also referred to herein as a VDAC2 mutein polypeptide, an anti-apoptotic VDAC2 mutein (i.e., a VDAC2 mimic), a pro-apoptotic VDAC2 mutein (i.e., a VDAC2/BAK inhibitor compound). A VDAC2 mutein is a polypeptide having an amino acid sequence that includes at least a fragment of a wild type (wt) VDAC2 polypeptide, wherein one or more amino acid residues of the wt VDAC2 polypeptide is mutated (e.g., insertion, substitution and/or deletion). As used herein, “wild type (wt) VDAC2” includes any wild-type VDAC2 protein, whether native or recombinant, having the naturally occurring amino acid sequence of the full-length, native VDAC2, as shown in, e.g., U.S. Pat. No. 5,780,235; GenBank Accession Nos: NP_{—}003366; NP_{—}776911; NP_{—}112644; NP_{—}035825; P45880; B44422; Q9TT14; Q60930; P82013; P81155; CAB94971 1; BAB13474; AAF78964; AAF80116; AF80102; AAF73513; AAD40241; XP293180; NP_{—}035825 or Q60930.

For example, the wild type VDAC2 polypeptide includes at least 10, 25, 50, 100, 200, or 250 amino acids of the 294 amino-acid long polypeptide encoded by the amino acid sequence of SEQ ID NO: 1, wherein X (or Xaa) at residue 24 can be any amino acid, but is preferably alanine or valine. The amino acid sequence of SEQ ID NO: 1 is presented below:

(SEQ ID NO: 1, length: 294 amino acids)

MATHGQTCAR PMCIPPSYAD LGKXARDIFN KGFGFGLVKL
DVKTKSCSGV EFSTSGSSNT DTGKVTGTLE TKYKWCEYGL
TFTEKWNTDN TLGTEIAIED QICQGLKLTF DTTFSPNTGK
KSGKIKSSYK RECINLGCDV DFDFAGPAIH GSAVFGYEGW
LAGYQMTFDS AKSKLTRNNF AVGYRTGDFQ LHTNVNDGTE
FGGSIYQKVC EDLDTSVNLA WTSGTNCTRF GIAAKYQLDP
TASISAKVNN SSLIGVGYTQ TLRPGVKLTL SALVDGKSIN
AGGHKVGLAL ELEA

A wild type VDAC2 polypeptide also includes at least 10, 25, 50, 100, 200, or 250 amino acids of the 295 amino-acid long polypeptide encoded by the amino acid sequence of SEQ ID NO: 2. The amino acid sequence of SEQ ID NO: 2 is presented below:

(SEQ ID NO: 2, length: 295 amino acids)

MAECCVPVCP RPMCIPPPYA DLGKAARDIF NKGFGFGLVK
LDVKTKSCSG VEFSTSGSSN TDTGKVSGTL ETKYKWCEYG
LTFTEKWNTD NTLGTEIAIE DQICQGLKLT FDTTFSPNTG
KKSGKIKSAY KRECINLGCD VDFDFAGPAI HGSAVFGYEG
WLAGYQMTFD SAKSKLTRSN FAVGYRTGDF QLHTNVNNGT
EFGGSIYQKV CEDFDTSVNL AWTSGTNCTR FGIAAKYQLD
PTASISAKVN NSSLIGVGYT QTLRPGVKLT LSALVDGKSF
NAGGHKLGLA LELEA

A VDAC2 protein also includes the amino acid sequence KVSGTLETKY (SEQ ID NO:3), the amino acid sequence KYQLDPTASISAKV (SEQ ID NO:4), or a combination of both amino acid sequences.

No particular length is implied by the terms “peptide” or “mutein.” The term “peptide,” as used herein, includes a full-length protein or polypeptide, such as, for example, a full-length VDAC2 protein or a full-length BAK protein. In some embodiments, the VDAC2 peptide or VDAC2 mutein is less than 294 amino acids in length, e.g., less than or equal to 250, 200, 150, 100, 75, 50, 35, 25 or 15 amino acid in length. The VDAC2 peptides and VDAC2 muteins are polymers of L-amino acids, D-amino acids, or a combination of both. For example, the peptides are D retro-inverso peptides. The term “retro-inverso isomer” refers to an isomer of a linear peptide in which the direction of the sequence is reversed and the chirality of each amino acid residue is inverted. See, e.g., Jameson et al., Nature, 368, 744-746 (1994); Brady et al., Nature, 368, 692-693 (1994). The net result of combining D-enantiomers and reverse synthesis is that the positions of carbonyl and amino groups in each amide bond are exchanged, while the position of the side-chain groups at each alpha carbon is preserved. Unless specifically stated otherwise, it is presumed that any given L-amino acid sequence of the invention may be made into an D retro-inverso peptide by synthesizing a reverse of the sequence for the corresponding native L-amino acid sequence.

In some embodiments, up to 20% or more of the residues may be so changed in the mutant or variant protein. Preferably, the anti-apoptotic VDAC2 muteins and the pro-apoptotic VDAC2 muteins are at least about 80% homologous to wt VDAC2, more preferably at least about 85%, 90%, 95%, 98%, and most preferably at least about 99% homologous to wt VDAC2. Any amino acid substitution, insertion, or deletion is encompassed by the invention. In favorable circumstances, the substitution is a conservative substitution as defined. Anti-apoptotic VDAC2 mutein-like activities include, for example, increased ability to bind BAK or preventing apoptosis, and pro-apoptotic VDAC2 mutein-like activities include, for example, a decreased ability to bind BAK or promoting apoptosis.

Suitable VDAC2 mimetics of the invention include, for example, an anti-apoptotic VDAC2 mutein, any of whose residues may be changed from wt VDAC2, wherein the anti-apoptotic VDAC2 mutein maintains VDAC2 protein biological activities (i.e. the ability to bind BAK) and physiological functions, or a functional fragment thereof. In general, a VDAC2-like variant that preserves anti-apoptotic VDAC2 mutein-like function includes any variant in which residues at a particular position in the sequence have been substituted by other amino acids, and further include the possibility of inserting an additional residue or residues between two residues of the parent protein as well as the possibility of deleting one or more residues from the parent sequence. In a VDAC2 mimetic, these mutations to the wt VDAC2 result in an anti-apoptotic VDAC2 mutein displaying an increased ability to form a complex with the BAK polypeptide, such that the BAK/VDAC2 complex is not disrupted in the presence of BH3 only molecules, such as BID, BIM, BAD or NOXA.

Suitable VDAC2/BAK inhibitor compounds include, for example, a pro-apoptotic VDAC2 mutein, any of whose residues may be changed from wt VDAC2, wherein the pro-apoptotic VDAC2 mutein has a decreased ability to bind BAK.

BH3 Domain Muteins

VDAC2/BAK inhibitor compounds of the invention include, for example, a mutated BH3 domain protein, also referred to herein as a BH3 domain mutein polypeptide, e.g., a BID mute in, a BIM mutein or a PUMA mutein. A BH3 domain mutein is a polypeptide having an amino acid sequence that includes at least a fragment of a wild type (wt) BH3 domain polypeptide (e.g., BID, BIM, PUMA), wherein one or more amino acid residues of the wt BH3 domain polypeptide is mutated (e.g., insertion, substitution and/or deletion). As used herein, “wild type (wt) BH3 domain polypeptide” includes any wild-type BH3 domain protein or isoform thereof, whether native or recombinant, having the naturally occurring amino acid sequence of the full-length, native BH3 domain polypeptide. For example, a wild type BID protein the amino acid sequence, shown, e.g., in GenBank Accession Nos. P55957; CAG30275; CAG28531; AAH36364; AAH09197; NP_{—}001187; AAP97190; AAP50259; and AAC34365; wild type BIM proteins includes the amino acid sequence shown, for example in GenBank Accession No. AAC39594; AAC39593; AAR06908; AAQ99150; BAB78589; NP_{—}619528; AAH33694; NP619527; NP619530; BAB78591; NP_{—}619532; BAB78592; NP_{—}006529; NP_{—}619529; BAB78590; AAQ82546; NP_{—}996885; and AAQ62569. Wild type PUMA proteins includes the amino acid sequence presented in GenBank Accession No. AAK31316; AAK39542; AAK39543; and NP_{—}055232.

No particular length is implied by the terms “peptide” or “mutein.” The term “peptide,” as used herein, includes a full-length protein or polypeptide, such as, for example, a full-length BH3 domain protein. In some embodiments, the BH3 domain peptide or BH3 domain mutein is less than 250 amino acids in length, e.g., less than or equal to 250, 200, 150, 100, 75, 50, 35, 25 or 15 amino acid in length. The BH3 domain peptides and BH3 domain muteins are polymers of L-amino acids, D-amino acids, or a combination of both. For example, the peptides are D retro-inverso peptides. The term “retro-inverso isomer” refers to an isomer of a linear peptide in which the direction of the sequence is reversed and the chirality of each amino acid residue is inverted. See, e.g., Jameson et al., Nature, 368, 744-746 (1994); Brady et al., Nature, 368, 692-693 (1994). The net result of combining D-enantiomers and reverse synthesis is that the positions of carbonyl and amino groups in each amide bond are exchanged, while the position of the side-chain groups at each alpha carbon is preserved. Unless specifically stated otherwise, it is presumed that any given L-amino acid sequence of the invention may be made into an D retro-inverso peptide by synthesizing a reverse of the sequence for the corresponding native L-amino acid sequence.

In some embodiments, up to 20% or more of the residues may be so changed in the mutant or variant protein. Preferably, the BH3 domain muteins are at least about 80% homologous to the wt sequence of the BH3 domain polypeptide, more preferably at least about 85%, 90%, 95%, 98%, and most preferably at least about 99% homologous to the wt sequence. Any amino acid substitution, insertion, or deletion is encompassed by the invention. In favorable circumstances, the substitution is a conservative substitution as defined.

Suitable VDAC2/BAK inhibitor compounds include, for example, a pro-apoptotic BH3 domain mutein, any of whose residues may be changed from the wt BH3 domain sequence, wherein the pro-apoptotic BH3 domain mutein interacts with VDAC2 while VDAC2 is associated with BAK in a VDAC2/BAK complex such that BAK is released from the VDAC2/BAK complex. For example, the BH3 mutein binds VDAC2 with a higher affinity or higher avidity than the wt BH3 domain polypeptide.

Methods of Screening for Anti-Apoptotic Compounds or VDAC2/BAK Inhibitor Compounds

The invention further provides a method of screening for anti-apoptotic compound or VDAC2/BAK inhibitor compounds. Any screening methods known to those skilled in the art can are used to identify compounds that act as anti-apoptotic compounds or inhibitor compounds.

The anti-apoptotic compounds or VDAC2/BAK inhibitor compounds of the invention are obtained using any of the numerous approaches in combinatorial library methods known in the art, such as, for example, biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds. See, e.g., Lam, 1997. Anticancer Drug Design 12: 145. These screening methods allow for rapid screening of a large number of candidate compounds.

A “small molecule,” as used herein, refers, for example, to a protein or peptide that has a molecular weight of less than about 5 kD and preferably less than about 4 kD, or to an inorganic or organic compound having a molecular weigh of less than about 600 Daltons. Small molecules are, for example, nucleic acids, peptides, polypeptides, peptidomimetics, carbohydrates, lipids or other organic or inorganic molecules. Libraries of chemical and/or biological mixtures, such as fungal, bacterial, or algal extracts, are known in the art and can be screened with any of the assays of the invention.

Examples of methods for the synthesis of molecular libraries can be found in the art, for example in: DeWitt, et al., 1993. Proc. Natl. Acad. Sci. U.S.A. 90: 6909; Erb, et al., 1994. Proc. Natl. Acad. Sci. U.S.A. 91: 11422; Zuckermann, et al., 1994. J. Med Chem. 37: 2678; Cho, et al., 1993. Science 261: 1303; Carrell, et al., 1994. Angew. Chem. Int. Ed. Engl. 33: 2059; Carell, et al., 1994. Angew. Chem. Int. Ed. Engl. 33: 2061; and Gallop, et al., 1994. J. Med Chem. 37:1233.

Libraries of compounds are presented in solution (e.g., Houghten, 1992. Biotechniques 13: 412421), on beads (Lam, 1991. Nature 354: 82-84), on chips (Fodor, 1993. Nature 364: 555-556), on bacteria (Ladner, U.S. Pat. No. 5,223,409), on spores (Ladner, U.S. Pat. No. 5,233,409), on plasmids (Cull, et al., 1992. Proc. Natl. Acad. Sci. USA 89: 1865-1869) or on phage (Scott and Smith, 1990. Science 249: 386-390; Devlin, 1990. Science 249: 404-406; Cwirla, et al., 1990. Proc. Natl. Acad. Sci U.S.A. 87: 6378-6382; Felici, 1991. J. Mol. Biol. 222: 301-310; Ladner, U.S. Pat. No. 5,233,409.).

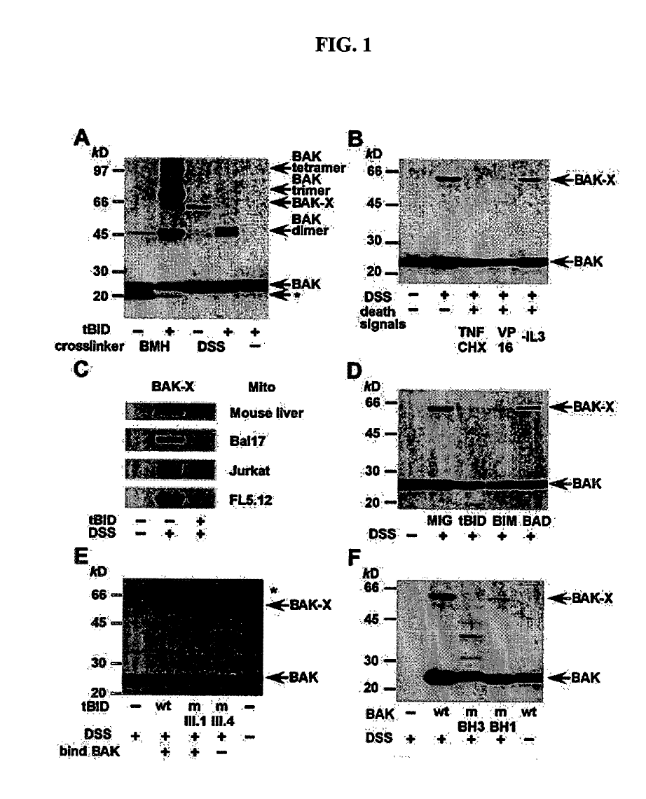

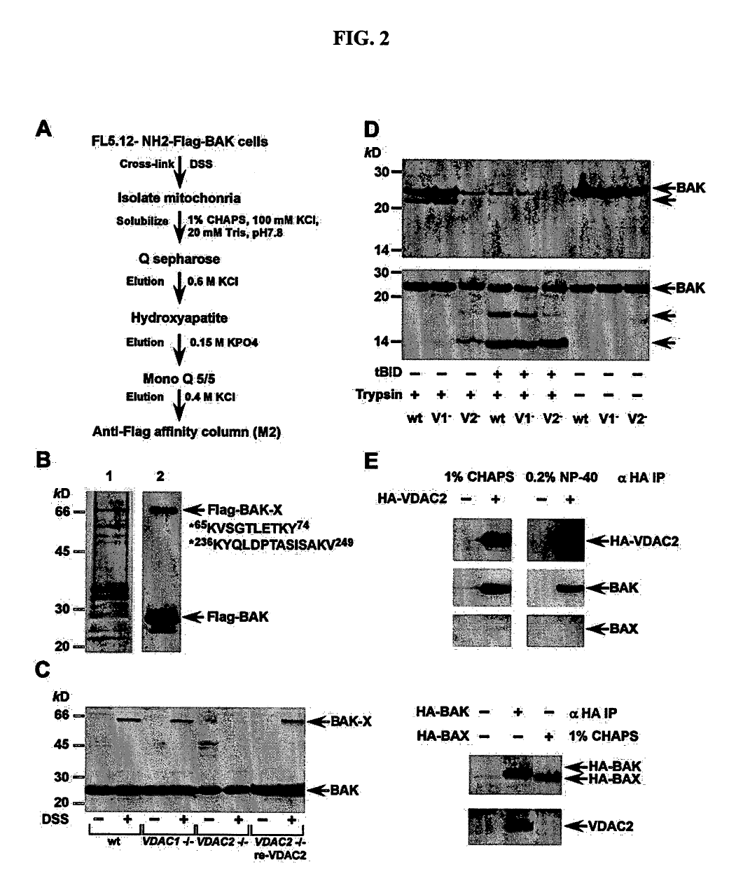

For example, pro-apoptotic compounds, such as VDAC2/BAK inhibitor compounds, are identified using a yeast two-hybrid system, a yeast genetic screening method developed for specifically identifying protein-protein interactions in an in vivo system. See e.g., FIG. 1, U.S. Pat. Nos. 5,468,614 and 5,283,173, each herein incorporated by reference in its entirety. See also Yang et al. (1995) Nucleic Acid Research 23, 1152-1156), incorporated herein by reference in its entirety. The yeast Two-Hybrid system relies on the interaction of two fusion proteins (also referred to as hybrids) to bring about the transcriptional activation of a reporter gene such as E. coli derived β-galactosidase (Lac Z). One fusion protein comprises a preselected protein fused to the DNA binding domain of a known transcription factor (e.g., GAL4). The second fusion protein comprises a polypeptide from a cDNA library fused to a transcriptional activation domain. The reporter gene is activated, when a polypeptide from the cDNA library binds the preselected target protein. Yeast cells harboring an activated reporter gene can be differentiated from other cells and the cDNA encoding for the interacting polypeptides can be easily isolated and sequenced.

Pro-apoptotic compounds or VDAC2/BAK inhibitor compounds are identified using a yeast three-hybrid system, a genetic screening system developed for detecting ligand-receptor interactions, RNA-protein interactions or multiple protein interactions in vivo (See e.g., Proc. Natl. Acad. Sci., 93, 8496-8501 (1996); Proc. Natl. Acad. Sci., 93, 12817 (1996); Nucl. Acids. Res., 27(4): 919-29 (1999); U.S. Pat. No. 5,928,868, these references are hereby incorporated by reference in their entirety). This system, which is adapted from the yeast two-hybrid system, includes a third synthetic hybrid ligand that acts as bridge to bring together the two fusion proteins of the yeast two-hybrid system. The reporter gene is activated the first and second fusion proteins are brought into proximity by the hybrid ligand (i.e., by the hybrid ligand binds both the first and second fusion proteins).

VDAC2/BAK inhibitor compounds are also identified using a reverse yeast two-hybrid system, a modified yeast two-hybrid system that identifies mutations, peptides or other small molecules that dissociate macromolecular interactions. (See e.g., Nucl. Acids. Res., 27(4): 919-29 (1999); Proc. Natl. Acad. Sci. USA, 93:10315-10320 (1996); Proc. Natl. Acad. Sci. USA, 93:10321-10326 (1996); TIBTECH, 17:374-381 (1999), each of these references are hereby incorporated by reference in their entirety). In this system, a toxic marker, such as, for example URA3 and CYH2, is used as a reporter gene, and the two fusion proteins are known to interact with each other. In the reverse yeast two-hybrid system, interaction between the hybrid proteins activates the toxic marker gene, and, as a result, the yeast colonies do not grow. Inhibitors of this interaction are detected by selecting the growing yeast colonies in which the hybrids have failed to interact.

Anti-apoptotic compounds (e.g., VDAC2 mimetics or VDAC2/BAK stabilizer compounds) or pro-apoptotic compounds (e.g., VDAC2/BAK inhibitor compounds) are identified using high throughput screening of libraries, such as, for example, random small molecule libraries. In these methods, candidate anti-apoptotic compounds promote interaction between BAK and endogenous VDAC2 or a VDAC2 mimetic, thereby preventing homo-oligomerization of BAK and induction of apoptosis. Candidate pro-apoptotic VDAC2/BAK inhibitor compounds disrupt the interaction between endogenous VDAC2 and BAK, thereby allowing BAK to form a homo-oligomer, which induces apoptosis. Monitoring the effect of the candidate compounds on the interaction between VDAC2 and BAK, in the presence and absence of a death stimulus, is accomplished, for example, by tagging VDAC2 and BAK with a detectable marker that indicates interaction between VDAC2 and BAK to form a VDAC2/BAK complex. Suitable detectable markers include, for example, fluorescence resonance energy (FRET) pairs such as CFP and YFP.

For example, CFP-VDAC2 and YFP-BAK are expressed in a population of cells (e.g., a population of mammalian cells or yeast cells) in the presence and absence of a death stimulus. Apoptosis is induced in the cells, for example, by using an inducible vector that is driven by a BH3-only molecule (e.g., tBID) or any of a variety of death signals (e.g., staurosporine, etoposide, UV irradiation). In these screening methods, compounds that prevent dissociation of CFP-VDAC2/YFP-BAK indicate that the candidate compound is an anti-apoptotic compound of the invention. Compounds that disrupt the interaction between CFP-VDAC2 and YFP-BAK indicate that the candidate compound is a VDAC2/BAK inhibitor compound of the invention.

The anti-apoptotic or pro-apoptotic compounds that are VDAC2/BAK stabilizer or inhibitor compounds are identified by contacting a cell population with a candidate compound. The cells may be engineered cells. For example, anti-apoptotic compounds or VDAC2/BAK inhibitor compounds are identified using a population of yeast cells that express human VDAC2 and human BAK at tolerated levels. The human VDAC2 and human BAK are introduced, for example, into porin 1, 2 null yeast cells, and apoptosis is induced in the yeast cells using, for example, an inducible vector driving expression of a BH3-only molecule (e.g., tBID). The yeast cells are contacted with a variety of candidate compounds. A decrease in the level of apoptosis in the cell population, when compared to the normal, control level of apoptosis in that cell population, indicates that the candidate compound is an anti-apoptotic compound, such as a VDAC2 mimetic or a VDAC2/BAK stabilizer compound. An increase in the level of apoptosis of the cell population, when compared to the normal, control level of apoptosis in that cell type (or cell population), indicates that the candidate compound is a pro-apoptotic compound, such as a VDAC2/BAK inhibitor compound.

The cell is contacted in vivo or in vitro. Suitable cells for use with the methods of the present invention include, for example, stromal, epithelial, organ specific, or blood-derived cells. For example, the cell types may be differentiated fibroblasts and keratinocytes. In other embodiments, the cell types can be fibroblasts, keratinocytes (including outer root sheath cells), melanocytes, endothelial cells, pericytes, monocytes, lymphocytes (including plasma cells), thrombocytes, mast cells, adipocytes, muscle cells, hepatocytes, neurons, nerve or neuroglia cells, osteocytes, osteoblasts, corneal epithelial cells, chondrocytes, ectodermal cells, endodermal cells, mesodermal cells and/or adult or embryonic stem cells. Suitable cell populations for identifying anti-apoptotic compounds and VDAC2/BAK inhibitor compounds also include mammalian cells that are BAX-null (i.e., do not express BAX, but do express BAK). Thus, in all screening assays using such BAX-null mammalian cells, intrinsic pathway cell death would be BAK-dependent.

The cell population includes at least one cell that has a VDAC2 protein and a BAK protein. The level of apoptosis in the presence of the candidate compound, with or without a death stimulus, is determined. A decrease in the level of apoptosis in the cell population, when compared to the normal, control level of apoptosis in that cell population, indicates that the candidate compound is a VDAC2/BAK stabilizer compound. An increase in the level of apoptosis of the cell population, when compared to the normal, control level of apoptosis in that cell type (or cell population), indicates that the candidate compound is a VDAC2/BAK inhibitor compound. The cell can be contacted in vivo or in vitro. The term, “normal, control level of apoptosis” refers to the level of apoptosis of a given cell (or population of cells) under standard cell culture techniques known in the art. The normal, control level of apoptosis can be determined using any of the methods described above.

Preparation of VDAC2 Polypeptides and VDAC2 Muteins

VDAC2 peptides and VDAC2 mutein polypeptides are prepared using modern cloning techniques, or may be synthesized by solid state methods by site-directed mutagenesis. A VDAC2 peptide and VDAC2 mutein polypeptide may include dominant negative forms of a polypeptide. Native VDAC2 peptides and VDAC2 mutein polypeptides are isolated from cells or tissue sources using standard protein purification techniques. Alternatively, VDAC2 peptides or VDAC2 mutein polypeptides are produced by recombinant DNA techniques. Optionally, VDAC2 peptides or VDAC2 mutein polypeptides are synthesized chemically using standard peptide synthesis techniques.

An “isolated” or “purified” protein or biologically active portion thereof is substantially free of cellular material or other contaminating proteins from the cell or tissue source from which the VDAC2 peptide and VDAC2 mutein polypeptide is derived, or substantially free from chemical precursors or other chemicals when chemically synthesized. The language “substantially free of cellular material” includes preparations of VDAC2 peptides and VDAC2 mutein polypeptides in which the protein is separated from cellular components of the cells from which it is isolated or recombinantly produced. In one embodiment, the language “substantially free of cellular material” includes preparations of VDAC2 peptides and VDAC2 mutein polypeptides having less than about 30% (by dry weight) of non-VDAC2 peptide and VDAC2 mutein polypeptide (also referred to herein as a “contaminating protein”), more preferably less than about 20% of non-VDAC2 peptide and VDAC2 mutein polypeptide, still more preferably less than about 10% of non-VDAC2 peptide and VDAC2 mutein polypeptide, and most preferably less than about 5% non-VDAC2 peptide and VDAC2 mutein polypeptide. When the VDAC2 peptide and VDAC2 mutein polypeptide or biologically active portion thereof is recombinantly produced, it is also preferably substantially free of culture medium, i.e., culture medium represents less than about 20%, more preferably less than about 10%, and most preferably less than about 5% of the volume of the protein preparation.

The language “substantially free of chemical precursors or other chemicals” includes preparations of VDAC2 peptides and VDAC2 mutein polypeptides in which the protein is separated from chemical precursors or other chemicals that are involved in the synthesis of the protein. In one embodiment, the language “substantially free of chemical precursors or other chemicals” includes preparations of VDAC2 peptides and VDAC2 mutein polypeptide having less than about 30% (by dry weight) of chemical precursors or non-VDAC2 peptide and VDAC2 mutein polypeptide chemicals, more preferably less than about 20% chemical precursors or non-VDAC2 peptide and VDAC2 mutein polypeptide chemicals, still more preferably less than about 10% chemical precursors or non-VDAC2 peptide and VDAC2 mutein polypeptide chemicals, and most preferably less than about 5% chemical precursors or non-VDAC2 peptide and VDAC2 mutein polypeptide chemicals.

The term “biologically equivalent” is intended to mean that the compositions of the present invention are capable of demonstrating some or all of the same apoptosis modulating effects although not necessarily to the same degree as the VDAC2 polypeptide deduced from sequences identified from cDNA libraries of human, rat or mouse origin or produced from recombinant expression symptoms.

By “substantially homologous” it is meant that the degree of homology of human, rat and mouse VDAC2 peptides and VDAC2 mutein polypeptides to a VDAC2 peptide and VDAC2 mutein polypeptide from any species is greater than that between VDAC2 peptides and VDAC2 mutein polypeptides and any previously reported member of the VDAC family of proteins.

Percent conservation is calculated from the above alignment by adding the percentage of identical residues to the percentage of positions at which the two residues represent a conservative substitution (defined as having a log odds value of greater than or equal to 0.3 in the PAM250 residue weight table). Conservation is referenced to sequences as indicated above for identity comparisons. Conservative amino acid changes satisfying this requirement are: R-K; E-D, Y-F, L-M; V-I, Q-H.

VDAC2 peptides and VDAC2 mutein polypeptides can also include derivatives of VDAC2 peptides and VDAC2 mutein polypeptides which are intended to include hybrid and modified forms of VDAC2 peptides and VDAC2 mutein polypeptide including fusion proteins and VDAC2 peptide and VDAC2 mutein polypeptide fragments and hybrid and modified forms in which certain amino acids have been deleted or replaced and modifications such as where one or more amino acids have been changed to a modified amino acid or unusual amino acid and modifications such as glycosylation so long as the hybrid or modified form retains the biological activity of VDAC2 peptides and VDAC2 mutein polypeptides. By retaining the biological activity, it is meant that cell death is induced by the VDAC2 polypeptide, although not necessarily at the same level of potency as that of the naturally-occurring VDAC2 polypeptide identified for human or mouse and that can be produced, for example, recombinantly. The difference in the level of potency for the VDAC2 mutein, as compared to the potency for the naturally-occurring VDAC2 polypeptide, is, for example, a one-fold, two fold, three fold, etc. difference. The terms induced and stimulated are used interchangeably throughout the specification. Alternatively, by retaining the biological activity, it is meant that cell death is prevented by the VDAC2 mutein polypeptide when compared to the cell death inducible ability of the naturally-occurring VDAC2 polypeptide identified for human or mouse and that can be produced, for example, recombinantly. The terms prevented and inhibited are used interchangeably throughout the specification.

Preferred variants are those that have conservative amino acid substitutions made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains ( e.g., tyrosine, phenylalanine, tryptophan, histidine). Thus, a predicted nonessential amino acid residue in a VDAC2 polypeptide is replaced with another amino acid residue from the same side chain family. Alternatively, in another embodiment, mutations can be introduced randomly along all or part of a VDAC2 coding sequence, such as by saturation mutagenesis, and the resultant mutants can be screened to identify mutants that retain activity.

Also included within the meaning of substantially homologous is any VDAC2 peptide and VDAC2 mutein polypeptide which may be isolated by virtue of cross-reactivity with antibodies to the VDAC2 peptide and VDAC2 mutein polypeptide described herein or whose encoding nucleotide sequences including genomic DNA, mRNA or cDNA may be isolated through hybridization with the complementary sequence of genomic or subgenomic nucleotide sequences or cDNA of the VDAC2 and VDAC2 mutein polynucleotides herein or fragments thereof. It will also be appreciated by one skilled in the art that degenerate DNA sequences can encode human VDAC2 and VDAC2 mutein polynucleotide sequences and these are also intended to be included within the present invention as are allelic variants of VDAC2 and VDAC2 mutein.

Chimeric and Fusion Proteins

The invention also provides VDAC2 chimeric or fusion proteins. As used herein, a VDAC2 or VDAC2 mutein “chimeric protein” or “fusion protein” comprises a VDAC2 peptide or VDAC2 mutein polypeptide operatively linked to a non-VDAC2 polypeptide. A “VDAC2 peptide” refers to a polypeptide having an amino acid sequence corresponding to a VDAC2 peptide whereas a “non-VDAC2 peptide refers to a polypeptide having an amino acid sequence corresponding to a protein that is not substantially homologous to the VDAC2 peptide, e.g., a protein that is different from the VDAC2 peptide and that is derived from the same or a different organism. Within a VDAC2 fusion peptide, the VDAC2 peptide can correspond to all or a portion of a VDAC2 peptide. In one embodiment, a VDAC2 peptide fusion protein comprises at least one biologically active portion of a VDAC2 peptide. In another embodiment, a VDAC2 peptide fusion protein comprises at least two biologically active portions of a VDAC2 peptide. Within the fusion protein, the term “operatively linked” is intended to indicate that the VDAC2 peptide and the non-VDAC2 peptide are fused in-frame to each other. The non-VDAC2 peptide can be fused to the N-terminus or C-terminus of the VDAC2 peptide.

For example, in on aspect the invention provides a chimeric peptide that include a first domain containing VDAC2 peptide or VDAC2 mutein operably linked to a second domain containing a translocation sequence.

A “translocation sequence” refers to any sequence of amino acids that directs a peptide in which it is present to a desired cellular destination. For example the translocation sequence is polyarginine. Thus, the translocation sequence can direct or facilitate penetration of the peptide across a biological membrane, e.g., a phospholipid membrane, mitochondrial membrane, or nuclear membrane. For example the translocation sequence directs the peptide from outside the cell, through the plasma membrane, and into the cytoplasm or to a desired location within the cell, e.g., the nucleus, the ribosome, the mitochondria, the ER, a lysosome, or peroxisome. Alternatively, or in addition, the translocation sequence can direct the peptide across a physiological barrier such as the blood-brain barrier, the trans-mucosal barrier, or the hematoencephalic, hematoretinal, gastrointestinal and pulmonary barriers.

Alternatively, a VDAC2 peptide or VDAC2 mutein fusion protein comprises a VDAC2 peptide or VDAC2 mutein operably linked to the extracellular domain of a second protein. In another embodiment, the fusion protein is a GST-VDAC2 peptide or GST-VDAC2 mutein fusion protein in which the VDAC2 peptide or VDAC2 mutein sequences are fused to the C-terminus of the GST (i.e., glutathione S-transferase) sequences. In another embodiment, the fusion protein is a VDAC2 peptide- or VDAC2 mutein -immunoglobulin fusion protein in which the VDAC2 peptide or VDAC2 mutein sequences comprising one or more domains are fused to sequences derived from a member of the immunoglobulin protein family. The VDAC2 peptide- or VDAC2 mutein -immunoglobulin fusion proteins of the invention can be incorporated into pharmaceutical compositions and administered to a subject. The VDAC2 peptide- or VDAC2 mutein -immunoglobulin fusion proteins of the invention can be used as immunogens to produce anti-VDAC2 antibodies in a subject, to purify VDAC2 peptide ligands, and in screening assays to identify molecules that inhibit the interaction of VDAC2 peptide with a BAK peptide ligand.

In another embodiment, the fusion protein is a VDAC2 peptide- or VDAC2 mutein-basic charged domain fusion protein in which the VDAC2 peptide or VDAC2 mutein sequences comprising one or more domains are fused to a basic peptide domain. The VDAC2 peptide- or VDAC2 mutein -basic charged domain fusion proteins of the invention can be incorporated into pharmaceutical compositions and administered to a subject. Several examples of biologically active fusion proteins, comprising basic peptide domains, for direct delivery of proteins into human patients in the context of protein therapy are known in the art, including, but not limited to, the human immunodeficiency virus type 1 (HIV-1) TAT protein, HIV-1 Rev protein, Drosophila Antennapedia or HIV-1 octaarginine protein. These basic peptide domains can be arginine-rich. These transducing proteins have been shown to have a membrane permeability and a carrier function for the delivery of proteins to the cytoplasm and nucleus of cells, both in vivo and in vitro. These cells can be mammalian cells (i.e. human cells). (Suzuki et al., J Biol Chem 276: 5836-40, 2001 and Suzuki et al., J Biol Chem 277: 2437-43, 2002).

A VDAC2 protein or VDAC2 mutein chimeric or fusion protein of the invention can be produced by standard recombinant DNA techniques. For example, DNA fragments coding for the different polypeptide sequences are ligated together in-frame in accordance with conventional techniques, e.g., by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers that give rise to complementary overhangs between two consecutive gene fragments that can subsequently be annealed and reamplified to generate a chimeric gene sequence (see, for example, Ausubel et al. (Eds.) CURRENT PROTOCOLS IN MOLECULAR BIOLOGY, John Wiley & Sons, 1992). Moreover, many expression vectors are commercially available that already encode a fusion moiety (e.g., a GST polypeptide). A VDAC2 peptide -encoding nucleic acid can be cloned into such an expression vector such that the fusion moiety is linked in-frame to the VDAC2 peptide.

VDAC2 Nucleic Acids

The present invention additionally relates to nucleic acids that encode VDAC2 peptides and/or VDAC2 mutein polypeptides. Nucleic acids encoding the VDAC2 peptides or VDAC2 muteins may be obtained by any method known in the art (e.g., by PCR amplification using synthetic primers hybridizable to the 3′- and 5′-termini of the sequence and/or by cloning from a cDNA or genomic library using an oligonucleotide sequence specific for the given gene sequence).

For recombinant expression of one or more VDAC2 peptides or VDAC2 muteins, the nucleic acid containing all or a portion of the nucleotide sequence encoding the peptide may be inserted into an appropriate expression vector (i.e., a vector that contains the necessary elements for the transcription and translation of the inserted peptide coding sequence). In some embodiments, the regulatory elements are heterologous (i.e., not the native gene promoter). Alternately, the necessary transcriptional and translational signals may also be supplied by the native promoter for the genes and/or their flanking regions.

A variety of host-vector systems may be utilized to express the peptide coding sequence(s). These include, but are not limited to: (i) mammalian cell systems that are infected with vaccinia virus, adenovirus, and the like; (ii) insect cell systems infected with baculovirus and the like; (iii) yeast containing yeast vectors or (iv) bacteria transformed with bacteriophage, DNA, plasmid DNA, or cosmid DNA. Depending upon the host-vector system utilized, any one of a number of suitable transcription and translation elements may be used.

Promoter/enhancer sequences within expression vectors may utilize plant, animal, insect, or fungus regulatory sequences, as provided in the invention. For example, promoter/enhancer elements can be used from yeast and other fungi (e.g., the GALA promoter, the alcohol dehydrogenase promoter, the phosphoglycerol kinase promoter, the alkaline phosphatase promoter). Alternatively, or in addition, they may include animal transcriptional control regions, e.g., (i) the insulin gene control region active within pancreatic β-cells (see, e.g., Hanahan, et al., 1985. Nature 315: 115-122); (ii) the immunoglobulin gene control region active within lymphoid cells (see, e.g., Grosschedl, et al., 1984. Cell 38: 647-658); (iii) the albumin gene control region active within liver (see, e.g., Pinckert, et al., 1987. Genes and Dev 1: 268-276; (iv) the myelin basic protein gene control region active within brain oligodendrocyte cells (see, e.g., Readhead, et al., 1987. Cell 48: 703-712); and (v) the gonadotropin-releasing hormone gene control region active within the hypothalamus (see, e.g., Mason, et al., 1986. Science 234: 1372-1378), and the like.

Expression vectors or their derivatives include, e.g. human or animal viruses (e.g., vaccinia virus, retrovirus or adenovirus); insect viruses (e.g., baculovirus); yeast vectors; bacteriophage vectors (e.g., lambda phage); plasmid vectors and cosmid vectors.

A host cell strain may be selected that modulates the expression of inserted sequences of interest, or modifies or processes expressed peptides encoded by the sequences in the specific manner desired. In addition, expression from certain promoters may be enhanced in the presence of certain inducers in a selected host strain; thus facilitating control of the expression of a genetically-engineered peptides. Moreover, different host cells possess characteristic and specific mechanisms for the translational and post-translational processing and modification (e.g., glycosylation, phosphorylation, and the like) of expressed peptides. Appropriate cell lines or host systems may thus be chosen to ensure the desired modification and processing of the foreign peptide is achieved. For example, peptide expression within a bacterial system can be used to produce an unglycosylated core peptide; whereas expression within mammalian cells ensures “native” glycosylation of a heterologous peptide.

Also included in the invention are derivatives, fragments, homologs, analogs and variants of VDAC2 peptides or VDAC2 muteins and nucleic acids encoding these peptides. For nucleic acids, derivatives, fragments, and analogs provided herein are defined as sequences of at least 6 (contiguous) nucleic acids, and which have a length sufficient to allow for specific hybridization. For amino acids, derivatives, fragments, and analogs provided herein are defined as sequences of at least 4 (contiguous) amino acids, a length sufficient to allow for specific recognition of an epitope.

The length of the fragments is less than the length of the corresponding full-length nucleic acid or polypeptide from which the VDAC2 peptides or VDAC2 muteins, or nucleic acid encoding same, is derived. Derivatives and analogs may be full length or other than full length, if the derivative or analog contains a modified nucleic acid or amino acid. Derivatives or analogs of the VDAC2 peptides or VDAC2 muteins include, e.g., molecules including regions that are substantially homologous to the peptides, in various embodiments, by at least about 30%, 50%, 70%, 80%, or 95%, 98%, or even 99%, identity over an amino acid sequence of identical size or when compared to an aligned sequence in which the alignment is done by a computer homology program known in the art. For example sequence identity can be measured using sequence analysis software (Sequence Analysis Software Package of the Genetics Computer Group, University of Wisconsin Biotechnology Center, 1710 University Avenue, Madison, Wis. 53705), with the default parameters therein.

In the case of polypeptide sequences, which are less than 100% identical to a reference sequence, the non-identical positions are preferably, but not necessarily, conservative substitutions for the reference sequence. Conservative substitutions typically include substitutions within the following groups: glycine and alanine; valine, isoleucine, and leucine; aspartic acid and glutamic acid; asparagine and glutamine; serine and threonine; lysine and arginine; and phenylalanine and tyrosine. Thus, included in the invention are peptides having mutated sequences such that they remain homologous, e.g. in sequence, in function, and in antigenic character or other function, with a protein having the corresponding parent sequence. Such mutations can, for example, be mutations involving conservative amino acid changes, e.g., changes between amino acids of broadly similar molecular properties. For example, interchanges within the aliphatic group alanine, valine, leucine and isoleucine can be considered as conservative. Sometimes substitution of glycine for one of these can also be considered conservative. Other conservative interchanges include those within the aliphatic group aspartate and glutamate; within the amide group asparagine and glutamine; within the hydroxyl group serine and threonine; within the aromatic group phenylalanine, tyrosine and tryptophan; within the basic group lysine, arginine and histidine; and within the sulfur-containing group methionine and cysteine. Sometimes substitution within the group methionine and leucine can also be considered conservative. Preferred conservative substitution groups are aspartate-glutamate; asparagine-glutamine; valine-leucine-isoleucine; alanine-valine; phenylalanine- tyrosine; and lysine-arginine.

Where a particular polypeptide is said to have a specific percent identity to a reference polypeptide of a defined length, the percent identity is relative to the reference peptide. Thus, a peptide that is 50% identical to a reference polypeptide that is 100 amino acids long can be a 50 amino acid polypeptide that is completely identical to a 50 amino acid long portion of the reference polypeptide. It might also be a 100 amino acid long polypeptide, which is 50% identical to the reference polypeptide over its entire length. Of course, other polypeptides will meet the same criteria.

The invention also encompasses allelic variants of the disclosed polynucleotides or peptides; that is, naturally-occurring alternative forms of the isolated polynucleotide that also encode peptides that are identical, homologous or related to that encoded by the polynucleotides. Alternatively, non-naturally occurring variants may be produced by mutagenesis techniques or by direct synthesis.

Species homologs of the disclosed polynucleotides and peptides are also provided by the present invention. “Variant” refers to a polynucleotide or polypeptide differing from the polynucleotide or polypeptide of the present invention, but retaining essential properties thereof. Generally, variants are overall closely similar, and in many regions, identical to the polynucleotide or polypeptide of the present invention. The variants may contain alterations in the coding regions, non-coding regions, or both.

In some embodiments, altered sequences include insertions such that the overall amino acid sequence is lengthened while the protein retains trafficking properties. Additionally, altered sequences may include random or designed internal deletions that shorten the overall amino acid sequence while the protein retains transport properties.

The altered sequences can additionally or alternatively be encoded by polynucleotides that hybridize under stringent conditions with the appropriate strand of the naturally-occurring polynucleotide encoding a polypeptide or peptide from which the VDAC2 peptide is derived. The variant peptide can be tested for BAK peptide -binding and modulation of BAK peptide -mediated activity using the herein described assays. ‘Stringent conditions’ are sequence dependent and will be different in different circumstances. Generally, stringent conditions can be selected to be about 5° C. lower than the thermal melting point (T_{M}) for the specific sequence at a defined ionic strength and pH. The T_{M} is the temperature (under defined ionic strength and pH) at which 50% of the target sequence hybridizes to a perfectly matched probe. Typically, stringent conditions will be those in which the salt concentration is at least about 0.02 molar at pH 7 and the temperature is at least about 60° C. As other factors may affect the stringency of hybridization (including, among others, base composition and size of the complementary strands), the presence of organic solvents and the extent of base mismatching, the combination of parameters is more important than the absolute measure of any one.

High stringency can include, e.g., Step 1: Filters containing DNA are pretreated for 8 hours to overnight at,65° C. in buffer composed of 6×SSC, 50 mM Tris-HCl (pH 7.5), 1 mM EDTA, 0.02% PVP, 0.02% Ficoll, 0.02% BSA, and 500 μg/ml denatured salmon sperm DNA. Step 2: Filters are hybridized for 48 hours at 65° C. in the above prehybridization mixture to which is added 100 mg/ml denatured salmon sperm DNA and 5−20×10⁶ cpm of ³²P-labeled probe. Step 3: Filters are washed for 1 hour at 37° C. in a solution containing 2×SSC, 0.01% PVP, 0.01% Ficoll, and 0.01% BSA. This is followed by a wash in 0.1×SSC at 50° C. for 45 minutes. Step 4: Filters are autoradiographed. Other conditions of high stringency that may be used are well known in the art. See, e.g., Ausubel et al., (eds.), 1993, CURRENT PROTOCOLS IN MOLECULAR BIOLOGY, John Wiley and Sons, NY; and Kriegler, 1990, GENE TRANSFER AND EXPRESSION, A LABORATORY MANUAL, Stockton Press, NY.

Moderate stringency conditions can include the following: Step 1: Filters containing DNA are pretreated for 6 hours at 55° C. in a solution containing 6×SSC, 5×Denhardt's solution, 0.5% SDS and 100 mg/ml denatured salmon sperm DNA. Step 2: Filters are hybridized for 18-20 hours at 55° C. in the same solution with 5−20×10⁶ cpm ³²P-labeled probe added. Step 3: Filters are washed at 37° C. for 1 hour in a solution containing 2×SSC, 0.1% SDS, then washed twice for 30 minutes at 60° C. in a solution containing 1×SSC and 0.1% SDS. Step 4: Filters are blotted dry and exposed for autoradiography. Other conditions of moderate stringency that may be used are well-known in the art. See, e.g., Ausubel et al., (eds.), 1993, CURRENT PROTOCOLS IN MOLECULAR BIOLOGY, John Wiley and Sons, NY; and Kriegler, 1990, GENE TRANSFER AND EXPRESSION, A LABORATORY MANUAL, Stockton Press, NY.

Low stringency can include: Step 1: Filters containing DNA are pretreated for 6 hours at 40° C. in a solution containing 35% formamide, 5×SSC, 50 mM Tris-HCl (pH 7.5), 5 mM EDTA, 0.1% PVP, 0.1% Ficoll, 1% BSA, and 500 μg/ml denatured salmon sperm DNA. Step 2: Filters are hybridized for 18-20 hours at 40° C. in the same solution with the addition of 0.02% PVP, 0.02% Ficoll, 0.2% BSA, 100 μg/ml salmon sperm DNA, 10% (wt/vol) dextran sulfate, and 5−20×10⁶ cpm ³²P-labeled probe. Step 3: Filters are washed for 1.5 hours at 55° C. in a solution containing 2×SSC, 25 mM Tris-HCl (pH 7.4), 5 mM EDTA, and 0.1% SDS. The wash solution is replaced with fresh solution and incubated an additional 1.5 hours at 60° C. Step 4: Filters are blotted dry and exposed for autoradiography. If necessary, filters are washed for a third time at 65-68° C. and reexposed to film. Other conditions of low stringency that may be used are well known in the art (e.g., as employed for cross-species hybridizations). See, e.g., Ausubel et al., (eds.), 1993, CURRENT PROTOCOLS IN MOLECULAR BIOLOGY, John Wiley and Sons, NY; and Kriegler, 1990, GENE TRANSFER AND EXPRESSION, A LABORATORY MANUAL, Stockton Press, NY.

VDAC2 Antibodies

Also included in the invention are antibodies to VDAC2 peptides or VDAC2 muteins or fragments thereof. The term “antibody” as used herein refers to immunoglobulin molecules and immunologically active portions of immunoglobulin (Ig) molecules, i.e., molecules that contain an antigen binding site that specifically binds (immunoreacts with) an antigen. Such antibodies include, but are not limited to, polyclonal, monoclonal, chimeric, single chain, F_{ab}, F_{ab}, and F_{(ab′)2} fragments, and an F_{ab} expression library. In general, an antibody molecule obtained from humans relates to any of the classes IgG, IgM, IgA, IgE and IgD, which differ from one another by the nature of the heavy chain present in the molecule. Certain classes have subclasses as well, such as IgG₁, IgG₂, and others. Furthermore, in humans, the light chain may be a kappa chain or a lambda chain. Reference herein to antibodies includes a reference to all such classes, subclasses and types of human antibody species.

An isolated VDAC2-related protein or an isolated VDAC2-related mutein of the invention may be intended to serve as an antigen, or a portion or fragment thereof, and additionally can be used as an immunogen to generate antibodies that immunospecifically bind the antigen, using standard techniques for polyclonal and monoclonal antibody preparation. The full-length protein can be used or, alternatively, the invention provides antigenic peptide fragments of the antigen for use as immunogens. An antigenic peptide fragment comprises at least 6 amino acid residues of the amino acid sequence of the full length protein, as shown in SEQ ID NO:1, and encompasses an epitope thereof such that an antibody raised against the peptide forms a specific immune complex with the full length protein or with any fragment that contains the epitope. By epitope reference is made to an antigenic determinant of a polypeptide. Typically, epitopes contain hydrophilic amino acids such that the particular region of the polypeptide is located on its surface and likely to be exposed in an aqueous based milieu. Preferably, the antigenic peptide comprises at least 3 amino acid residues in a spatial conformation which is unique to the epitope. Generally, the antigenic peptide comprises at least 5 amino acid residues, or at least 10 amino acid residues, or at least 15 amino acid residues, or at least 20 amino acid residues, or at least 30 amino acid residues. Furthermore, antibodies to a VDAC2 peptide or fragments thereof can also be raised against oligopeptides that include a conserved region such as the α6 helix domain of BID identified herein.

In certain embodiments of the invention, at least one epitope encompassed by the antigenic peptide is a region of VDAC2 that is located on the surface of the protein, e.g., a hydrophilic region. A hydrophobicity analysis of the human VDAC2 sequence will indicate which regions of a VDAC2 peptide are particularly hydrophilic and, therefore, are likely to encode surface residues useful for targeting antibody production. As a means for targeting antibody production, hydropathy plots showing regions of hydrophilicity and hydrophobicity may be generated by any method well known in the art, including, for example, the Kyte Doolittle or the Hopp Woods methods, either with or without Fourier transformation. See, e.g., Hopp and Woods, 1981, Proc. Nat. Acad. Sci. USA 78: 3824-3828; Kyte and Doolittle 1982, J. Mol. Biol. 157: 105-142, each of which is incorporated herein by reference in its entirety. Antibodies that are specific for one or more domains within an antigenic protein, or derivatives, fragments, analogs or homologs thereof, are also provided herein.

A protein of the invention, or a derivative, fragment, analog, homolog or ortholog thereof, may be utilized as an immunogen in the generation of antibodies that immunospecifically bind these protein components.

Various procedures known within the art may be used for the production of polyclonal or monoclonal antibodies directed against a protein of the invention, or against derivatives, fragments, analogs homologs or orthologs thereof (see, for example, Antibodies: A Laboratory Manual, Harlow E, and Lane D, 1988, Cold Spring Harbor Laboratory Press, Cold Spring Harbor, N.Y., incorporated herein by reference).

For the production of polyclonal antibodies, various suitable host animals (e.g., rabbit, goat, mouse or other mammal) may be immunized by one or more injections with the native protein, a synthetic variant thereof, or a derivative of the foregoing, and the antibodies can be isolated and purified using well known techniques in the art. An appropriate immunogenic preparation can contain, for example, the naturally occurring immunogenic protein, a chemically synthesized polypeptide representing the immunogenic protein, or a recombinantly expressed immunogenic protein. Furthermore, the protein may be conjugated to a second protein known to be immunogenic in the mammal being immunized. Examples of such immunogenic proteins include but are not limited to keyhole limpet hemocyanin, serum albumin, bovine thyroglobulin, and soybean trypsin inhibitor. The preparation can further include an adjuvant. Various adjuvants used to increase the immunological response include, but are not limited to, Freund's (complete and incomplete), mineral gels (e.g., aluminum hydroxide), surface active substances (e.g., lysolecithin, pluronic polyols, polyanions, peptides, oil emulsions, dinitrophenol, etc.), adjuvants usable in humans such as Bacille Calmette-Guerin and Corynebacterium parvum, or similar immunostimulatory agents. Additional examples of adjuvants which can be employed include MPL-TDM adjuvant (monophosphoryl Lipid A, synthetic trehalose dicorynomycolate) and CpG dinucleotide motifs (Krieg, A. M. Biochim Biophys Acta 1489(1):107-16, 1999).

Monoclonal antibodies to the VDAC2 peptides or VDAC2 muteins of the invention can be prepared using hybridoma methods, such as those described by Kohler and Milstein, Nature, 256:495 (1975); Goding, Monoclonal Antibodies: Principles and Practice, Academic Press, (1986) pp. 59-103; Kozbor, J. Immunol., 133:3001 (1984); Brodeur et al., Monoclonal Antibody Production Techniques and Applications, Marcel Dekker, Inc., New York, (1987) pp. 51-63; Munson and Pollard, Anal. Biochem., 107:220 (1980), the teachings of which are hereby incorporated by reference in their entirety.

The monoclonal antibodies can also be made by recombinant DNA methods, such as those described in U.S. Pat. No. 4,816,567; Morrison, Nature 368, 812-13 (1994)), the teachings of which are hereby incorporated by reference in their entirety.

The antibodies directed against the protein antigens of the invention can further comprise humanized antibodies or human antibodies. These antibodies are suitable for administration to humans without engendering an immune response by the human against the administered immunoglobulin. Humanized forms of antibodies are chimeric immunoglobulins, immunoglobulin chains or fragments thereof (such as Fv, Fab, Fab′, F(ab′)₂ or other antigen-binding subsequences of antibodies) that are principally comprised of the sequence of a human immunoglobulin, and contain minimal sequence derived from a non-human immunoglobulin. Humanization can be performed following the methods of Winter and co-workers (Jones et al., Nature, 321:522-525 (1986); Riechmann et al., Nature, 332:323-327 (1988); Verhoeyen et al., Science, 239:1534-1536 (1988)), U.S. Pat. No. 5,225,539; Jones et al., 1986; Riechmann et al., 1988; and Presta, Curr. Op. Struct. Biol., 2:593-596 (1992), the teachings of which are hereby incorporated by reference in their entirety.

Fully human antibodies relate to antibody molecules in which essentially the entire sequences of both the light chain and the heavy chain, including the CDRs, arise from human genes. Such antibodies are termed “human antibodies”, or “fully human antibodies” herein. Fully human monoclonal antibodies can be prepared by any of a variety of techniques known in the art. For example, fully human monoclonal antibodies can be produced using the trioma technique, the techniques of Kozbor, et al., 1983 Immunol Today 4: 72; Cole, et al., 1985 In: MONOCLONAL ANTBODIES AND CANCER THERAPY, Alan R. Liss, Inc., pp. 77-96; Cote, et al., 1983. Proc Natl Acad Sci USA 80: 2026-2030); Hoogenboom and Winter, J. Mol. Biol., 227:381 (1991); Marks et al., J. Mol. Biol., 222:581 (1991); Marks et al. (Bio/Technology 10, 779-783 (1992)); Lonberg et al. (Nature 368 856-859 (1994)); Morrison (Nature 368, 812-13 (1994)); Fishwild et al,( Nature Biotechnology 14, 845-51 (1996)); Neuberger (Nature Biotechnology 14, 826 (1996)); Lonberg and Huszar (Intern. Rev. Immunol. 13 65-93 (1995); PCT publications WO94/02602, WO 96/33735, WO 96/34096, and WO 99/53049; and in U.S. Pat. Nos. 5,545,807; 5,545,806; 5,569,825; 5,625,126; 5,633,425; 5,661,016; 5,916,771; and 5,939,598, the teachings of which are hereby incorporated by reference in their entirety.

According to the invention, techniques can be adapted for the production of single-chain antibodies specific to an antigenic protein of the invention (see e.g., U.S. Pat. No. 4,946,778). In addition, methods can be adapted for the construction of F_{ab} expression libraries (see e.g., Huse, et al., 1989 Science 246: 1275-1281) to allow rapid and effective identification of monoclonal F_{ab} fragments with the desired specificity for a protein or derivatives, fragments, analogs or homologs thereof. Antibody fragments that contain the idiotypes to a protein antigen may be produced by techniques known in the art including, but not limited to: (i) an F_{(ab′)2} fragment produced by pepsin digestion of an antibody molecule; (ii) an F_{ab} fragment generated by reducing the disulfide bridges of an F_{(ab′)2} fragment; (iii) an F_{ab} fragment generated by the treatment of the antibody molecule with papain and a reducing agent and (iv) Fᵥ fragments.

Bispecific antibodies are monoclonal, preferably human or humanized, antibodies that have binding specificities for at least two different antigens. In the present case, one of the binding specificities is for an antigenic protein of the invention. The second binding target is any other antigen, and advantageously is a cell-surface protein or receptor or receptor subunit. Methods for making bispecific antibodies are known in the art. (Milstein and Cuello, Nature, 305:537-539 (1983); Suresh et al., Methods in Enzymology, 121:210 (1986); Brennan et al., Science 229:81 (1985); Shalaby et al., J. Exp. Med. 175:217-225 (1992); Kostelny et al., J. Immunol. 148(5):1547-1553 (1992); Hollinger et al., Proc. Natl. Acad. Sci. USA 90:6444-6448 (1993); Gruber et al., J. Immunol. 152:5368 (1994); Tutt et al., J. Immunol. 147:60 (1991); WO 93/08829; WO 96/27011; and Traunecker et al., 1991 EMBO J., 10:3655-3659), the teachings of which are hereby incorporated by reference in their entirety.

Heteroconjugate antibodies are also within the scope of the present invention. Heteroconjugate antibodies are composed of two covalentlyjoined antibodies. Such antibodies have, for example, been disclosed in U.S. Pat. No. 4,676,980; WO 91/00360; WO 92/200373; EP 03089; and U.S. Pat. No. 4,676,980, the teachings of which are hereby incorporated by reference in their entirety.

It can be desirable to modify the antibody of the invention with respect to effector function, so as to enhance, e.g., the effectiveness of the antibody in treating cancer. Methods of engineering effector function are disclosed, for example, in Caron et al., J. Exp Med., 176: 1191-1195 (1992); Shopes, J. Immunol., 148: 2918-2922 (1992); Wolff et al. Cancer Research, 53: 2560-2565 (1993); and Stevenson et al., Anti-Cancer Drug Design, 3: 219-230 (1989), the teachings of which are hereby incorporated by reference in their entirety.

The invention also pertains to immunoconjugates comprising an antibody conjugated to a cytotoxic agent such as a chemotherapeutic agent, toxin (e.g., an enzymatically active toxin of bacterial, fungal, plant, or animal origin, or fragments thereof), or a radioactive isotope (i.e., a radioconjugate). (Vitetta et al., Science, 238: 1098 (1987); WO94/11026, the teachings of which are hereby incorporated by reference in their entirety).

In another embodiment, the antibody can be conjugated to a “receptor” (such streptavidin) for utilization in tumor pretargeting wherein the antibody-receptor conjugate is administered to the patient, followed by removal of unbound conjugate from the circulation using a clearing agent and then administration of a “ligand” (e.g., avidin) that is in turn conjugated to a cytotoxic agent.

Pharmaceutical Compositions

The compounds, e.g., VDAC2 peptides, VDAC2 mutein polypeptides, VDAC2 mimetics, VDAC2/BAK stabilizer compounds, VDAC2/BAK inhibitor compounds, nucleic acids encoding VDAC2 peptides and VDAC2 mutein polypeptides, and VDAC2 and VDAC2 mutein antibodies (also referred to herein as “active compounds”) of the invention, and derivatives, fragments, analogs and homologs thereof, can be incorporated into pharmaceutical compositions suitable for administration. Such compositions typically comprise the nucleic acid molecule, or protein, and a pharmaceutically acceptable carrier. As used herein, “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, finger's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., VDAC2 peptides, VDAC2 mutein polypeptides, VDAC2 mimetics, VDAC2/BAK stabilizer compounds, VDAC2/BAK inhibitor compounds, nucleic acids encoding VDAC2 peptides and VDAC2 mutein polypeptides, and VDAC2 and VDAC2 mutein antibodies) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811, incorporated fully herein by reference.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved.

The pharmaceutical compositions can be included in a container, pack, or dispenser together with instructions for administration.

The invention will be further described in the following examples, which do not limit the scope of the invention described in the claims.

### EXAMPLES

### Example 1

### Materials and General Methods

Plasmid Construction and Retrovirus Production

Murine vdac1, vdac2, N-terminal HA-tagged vdac2, N-terminal-HA tagged Bax or Bak, as well as wild type and mutant Bak (mBH1, W122A, G123E, R124A; mBH3, L75E) were cloned into the retroviral expression vector MSCV-IRES-GFP (pMIG). PcDNA3-tBID (wt and mutants) was described previously M. C. Wei et al., Genes Dev 14, 2060-71. (2000). Generation of amphotropic retroviruses and retroviral infection were described previously E. H. Cheng et al., Mol Cell 8, 705-11. (2001). Retroviral expression of each indicated protein was confirmed by immunoblot.

Purification of BAK-VDAC2 Cross-Linked Complex

All purification steps were carried out at 4° C. The chromatographic step of Hydroxyapatite (Calbiochem) was carried out using conventional stepwise chromatography. The chromatographic steps of Q-sepharose and Mono-Q were performed on an automatic fast protein liquid chromatography (FPLC) station (Pharmacia). FL5.12 cells expressing the N-terminal Flag-tagged BAK and BCL-2, treated with 5 mM DSS at room temperature for 30 min, were subjected to subcellular fractionation as described previously. Lysates (50 mg protein in buffer A-1% CHAPS, 100 mM KCl, 20 mM Tris, pH 7.8, supplemented with protease inhibitors) of heavy membrane fraction were applied onto a HiTrap Q Sepharose column (Pharmacia). The bound materials were step eluted with buffer A containing 0.2 M, 0.6 M, 0.8 M, and 1 M KCl. The Flag-BAK-X complex was followed by an immunoblot for BAK in various fractions. Flag-BAK-X was present predominantly in the 0.6 M KCl eluent which was subjected to dialysis against buffer A containing 10 mM potassium phosphate and then loaded onto a hydroxyapatite column (bed volume 5 ml) equilibrated with the same buffer. The bound materials were step eluted with buffer A containing 0.15 M or 0.5 M potassium phosphate. Most of the Flag-BAK-X complex was present in the 0.15 M potassium phosphate eluent, which was subjected to dialysis against buffer A, then loaded onto Mono Q (HR 5/5, Pharmnacia). The Flag-BAK-X complex was present predominantly in the 0.4 M KCl eluent, which was subjected to dialysis against buffer A, then loaded onto an anti-Flag (M2, Sigma) column. The bound proteins were eluted with Flag peptide (1 mg/ml in buffer A). Aliquots of eluent were analyzed by silver stain (BIO-RAD) and by an immunoblot developed with Anti-BAK Ab. The ˜60 kD band corresponding to Flag-BAK-X was excised from a silver stained 4-12 % NuPAGE gel, subjected to combined liquid chromatography and tandem mass spectrometry analysis (Harvard Medical School Taplin Biological Mass Spectrometry Facility).

Determination of BAK Conformation by Limited Proteolysis

Isolated mitochondria (50 μg) were incubated with 30 μg/ml trypsin in MRM buffer (250 mM sucrose, 10 mM Hepes, 1 mM ATP, 5 mM succinate, 0.08 mM ADP, 2 mM K2HPO4, 40 mM KCl, 1 mM MgC12, pH 7.5) on ice for 20 minutes. Soybean trypsin inhibitor (100 μg/ml) was added, and the mitochondria were pelleted and solubilized in SDS-PAGE buffer. Samples were analyzed by immunoblots using an anti-BAK NT Ab (Upstate Biotechnology) recognizing the N-terminal 34 amino acid residues, or the anti-BAK G23 Ab (Santa Cruz) reacts with a peptide of 20 amino acid residues spanning the BH3 domain.

Gel Filtration Analyses

The chromatographic step of Superdex 200 (HR 10/30, Amersham-Pharmacia) was performed on an automatic fast protein liquid chromatography (FPLC) station (Amersham-Pharmacia) at 4° C. The column was equilibrated with buffer containing 2% CHAPS, 300 mM NaCl, 0.2 mM DTT, and 25 mM Hepes, pH 7.5 and calibrated with thyroglobulin (669 kD, Amersham-Pharmacia), ferritin (440 kD, Amersham-Pharmacia), catalase (232 kD, Amersham-Pharmacia), aldolase (158 kD, Amersham-Pharmacia), bovine serum albumin (66 kD, Calbiochem), and cytochrome c (14 kD, Sigma). 200 μl of mitochondrial lysates (1 mg/ml protein in the same buffer as above) was loaded onto the column, eluted at a flow rate of 0.3 ml/minute. Fractions of 0.6 ml were collected, precipitated by trichloroacetic acid, and analyzed by an immunoblot (fractions 16-31) developed with an anti-BAK Ab.

Antibodies

Antibodies used for immunoblot included anti-BAK NT (Upstate Biotechnology), anti-BAK G23 (Santa Cruz), anti-cytochrome c (Pharmingen), anti-BCL-2 (/100, Pharmingen), anti-HA-Biotin (12CA5, Roche), anti-BAX (651), and anti-VDAC2. Antibodies used for immunoprecipitation included anti-HA (12CA5, Roche) and anti-BCL-2 (6C8).

Generation of vdac2 -/- Mouse Embryonic Fibroblasts

vdac1 -/- and vdac2 -/- embryonic stem (ES) cells were described previously S. Wu, M. J. Sampson, W. K. Decker, W. J. Craigen, Biochim Biophys Acta 1452, 68-78. (1999). 129/SvEv derived ES cell clones with homozygous deletion of vdac2 alleles were microinjected into blastocysts from C57BL/6 female mice. Mouse embryonic fibroblasts derived from E13.5 day chimeric embryos were treated with 0.3 mg/ml G418 (GIBCO-BRL) at second passage for 5 days to select for vdac2 -/- cells. Normally, 100% of MEFs died at 3 days in the presence of 0.3 mg/ml G418. Deficiency of VDAC2 in cells following G418 selection was confirmed by an immunoblot developed with an anti-VDAC2 Ab.

Fluorogenic Caspase Activity Assay

Cellular extracts from 10⁵ cells were incubated with DEVD-AFC at 37° C. for 1 hour. The release of fluorescent AFC was measured at excitation of 400 nm and emission of 505 nm using a luminescence spectrometer (LS50B, Perkin Elmer).

Flow Cytometric Analysis of Mitochondrial Membrane Potential

MEF cell lines with or without staurosporine treatment were harvested and incubated with PBS containing 150 nM tetramethylrhodamine ethyl ester (TMRE, Molecular Probes) at 37° C. for 30 minutes. Cells were pelleted and resuspended in PBS containing 15 nM TMRE. Samples were analyzed using a FACSCalibur flow cytometer (Becton Dickinson). The uncoupler carbonyl cyanide chlorophenylhydrazone (CCCP) was added subsequently to validate the specificity of TMRE staining in reflecting mitochondrial membrane potential.

Cytochrome c Release Assay

MEF cell lines treated with 1 μM staurosporine were harvested at various time points and lysed for 5 min in isotonic buffer containing 10 mM KCl, 5 mM MgCl₂, 1 mM EDTA, 1 mM EGTA, 250 mM sucrose, 20 mM Hepes, pH 7.2, 0.025% digitonin, and complete protease inhibitors (Roche). Organelles that contain mitochondria were removed from the soluble cytosolic fraction by pelleting at 15000 g. Supernatant and mitochondrial pellets were solubilized in SDS-PAGE loading buffer and analyzed by an immunoblot, developed with an anti-cytochrome c Ab. Protein cross-linking, immunoblot, immunoprecipitation, isolation of mitochondria, cell viability assays, and anti-cytochrome c immunohistochemistry were described previously E. H. Cheng et al., Mol Cell 8, 705-11. (2001).

### Example 2

### Screening for Candidate Proteins that Interact with BAK at the Mitochondria of Viable Cells

Protein crosslinkers were used to search for a candidate protein that interacts with BAK at the mitochondria in viable cells. A ˜60 kD mobility-shifted complex immunoreactive with an antibody (Ab) to BAK was detected after treatment of purified mitochondria (FIG. 1A) or viable whole cells (FIG. 1B) with the crosslinker disuccinimidyl suberate (DSS). This presumed complex between BAK and a candidate ˜35 kD interacting protein was designated “BAK-X.” The ˜60 kD BAK-X complex was distinct in size as compared to BAK homo-oligomers detected by bismaleimidohexane (BMH) crosslinking (FIG. 1A). The same sized BAK complex was detected in multiple cell lines and in normal liver prior to death stimuli (FIG. 1C). The BAK-X complex was lost when mitochondria were treated with tBID (FIG. 1A, C) or cells were treated with death stimuli including TNFα, etoposide, or withdrawal of survival factor (FIG. 1B). Expression of BH3-only members BAD, BIM, or tBID killed cells and diminished the amount of BAK-X complex (FIG. 1D). Mutant tBID molecules indicated that only BH3 domains (BH3 mutations mIII.1, not mIII.4) capable of binding BAK and initiating release of cytochrome c caused dissociation of the BAK-X complex (FIG. 1E) M. C. Wei et al., Genes Dev 14, 2060-71. (2000). In cells expressing BAK mutated within the BH3 domain (L75E) the BAK-X complex was not detected, and in cells expressing a mutated BH1 domain (W122A, G123E, R124A) the complex was substantially reduced (FIG. 1F). This finding suggested that the candidate protein, X, interacted with the BAK pocket formed by the BH1, BH2, and BH3 domains, and could be displaced, directly or indirectly, by BH3-only molecules.

### Example 3

### Isolation of the Candidate Protein “X” that Interacts with BAK to form the BAK-X Complex

To isolate the candidate protein X, N-terminal Flag-tagged BAK was expressed in a cell line to facilitate affinity purification. Serial steps of Q sepharose, hydroxyapatite, MonoQ, and anti-Flag affinity column chromatography (FIG. 2A) enriched the BAK-X complex from mitochondria (FIG. 2B). Liquid chromatography and tandem mass spectrometry analysis of the complex revealed the presence of two definitive tryptic fragments of VDAC2 (FIG. 2B). When vdac2-deficient embryonic stem (ES) cells were subjected to DSS crosslinking (S. Wu, M. J. Sampson, W. K. Decker, W. J. Craigen, Biochim Biophys Acta 1452, 68-78. (1999)), no 60 kD BAK-containing complex was detected (FIG. 2C), further implicating VDAC2, but not VDAC1 or VDAC3, as the BAK-interacting protein. Re-expressing VDAC2 in vdac2 -/- ES cells by retroviral transduction restored the 60 kD complex. The BAK complex was present in cells deficient for the closely related homolog VDAC1 (FIG. 2C). The active conformation of BAK displays more proteolytic susceptibility compared to the inactive conformer (FIG. 2D). The pattern of trypsin digested BAK from isolated mitochondria of wild-type (wt), vdac2-deficient or vdac1-deficient cells was then assessed. The absence of VDAC2 increased the susceptibility of BAK to proteolysis (FIG. 2D). Even prior to tBID exposure, a substantial portion of BAK in vdac2-deficient mitochondria demonstrated a pattern resembling activation (FIG. 2D). These data supported a model in which VDAC2, but not VDAC 1, regulates the conformation of BAK.

Endogenous BAK, but not BAX, was efficiently co-precipitated with HA-tagged VDAC2 (FIG. 2E). This interaction was noted following solubilization of cell extracts with the zwitterionic detergent CHAPS, which unlike nonionic detergents does not induce a conformational change in BAX or BAK Y. (T. Hsu, R. J. Youle, J Biol Chem. 273, 10777-83. (1998); X. Roucou, S. Montessuit, B. Antonsson, J. C. Martinou, Biochem J368, 915-21. (2002)). Solubilization of proteins in non-ionic NP40 reduced the amount of BAK complexed with VDAC2, and this finding was also consistent with VDAC2 preferentially interacting with inactive BAK. An HA-tagged BAK, but not HA-tagged BAX co-precipitated endogenous VDAC2 (FIG. 2E). No BCL-2 or BCL-X_{L} co-precipitated with HA-BAX or HA-BAK in 1% CHAPS, consistent with VDAC2, but not BCL-2 or BCL-X_{L}, binding to the inactive conformer of BAK.

### Example 4

### VDAC2 Modulation of Apoptosis Mediated by BAK or BAX

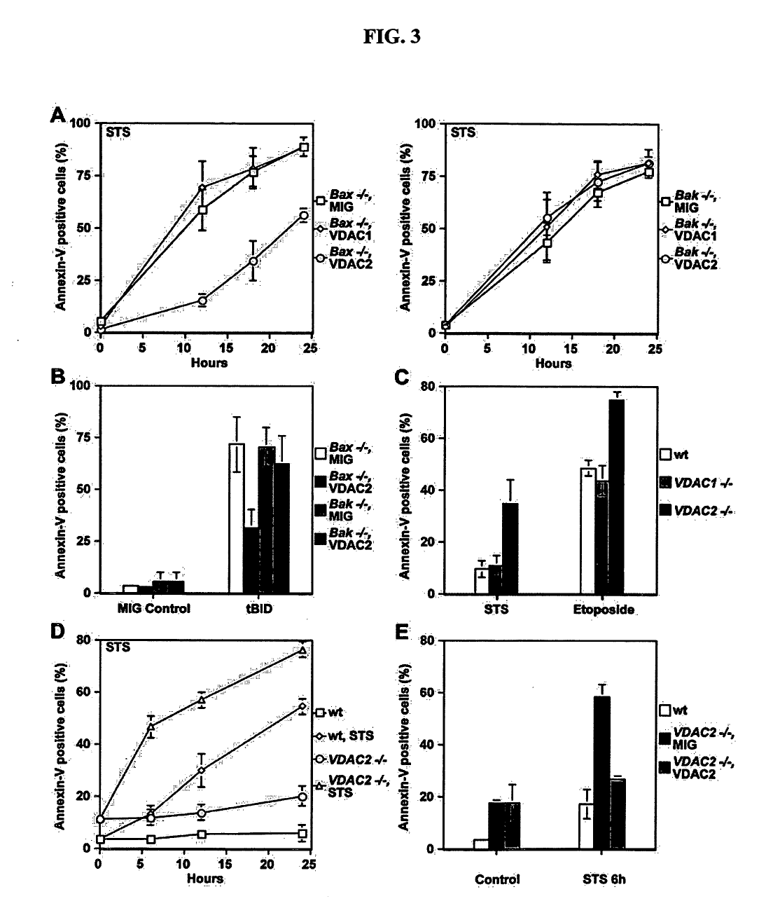

Previous studies have shown that cells doubly deficient for BAX and BAK are resistant to intrinsic death stimuli, whereas cells singly deficient for BAX or BAK are still susceptible (M. C. Wei et al., Science 292, 727-30. (2001)). As VDAC2 interacts with BAK but not BAX, the ability of VDAC2 to modulate apoptosis mediated by BAK or BAX was tested. According to one hypothesis, VDAC2 might function as an inhibitor of BAK or VDAC2 itself might function as a proapoptotic factor once released from BAK. To address these alternatives VDAC 1 or VDAC2 was expressed in Bax -/- or Bak -/- singly deficient mouse embryonic fibroblasts (MEFs) followed by treatment with staurosporine or etoposide. Expression of VDAC2 significantly inhibited apoptosis in Bax -/- cells that possess BAK, but had no effect in Bak -/- cells. In contrast, expression of VDAC1 displayed no substantial effect on cell viability (FIG. 3A and FIG. 6). tBID, an activated BH3-only molecule, activates BAX and BAK and requires one or the other to induce apoptosis (M. C. Wei et al., Science 292, 727-30. (2001)). Expression of VDAC2 inhibited tBID-induced apoptosis of Bax -/- cells but not Bak -/- cells, further supporting a selective role for VDAC2 in negatively regulating BAK-dependent apoptosis (FIG. 3B). Embryonic stem cells deficient for VDAC2 but not those deficient for VDAC1 proved more sensitive to various death stimuli including staurosporine and etoposide (FIG. 3C).

To consolidate these findings, vdac2 -/- MEFs, selected from chimeric embryos generated by microinjection of vdac2 -/- ES cells into wt blastocysts, were established. vdac2 -/- MEFs displayed more spontaneous apoptosis and proved more sensitive to staurosporine and etoposide (FIG. 3D and FIG. 8A), whereas vdac1 -/- or vdac3 -/- cells were similar to wt cells (FIG. 8B). Re-expression of VDAC2 to normal physiologic levels in vdac2 -/- MEFs reversed the increased susceptibility to apoptosis (FIG. 3E).

### Example 5

### Comparison of Apoptotic Hallmarks in vdac2 -/- and wt Cells

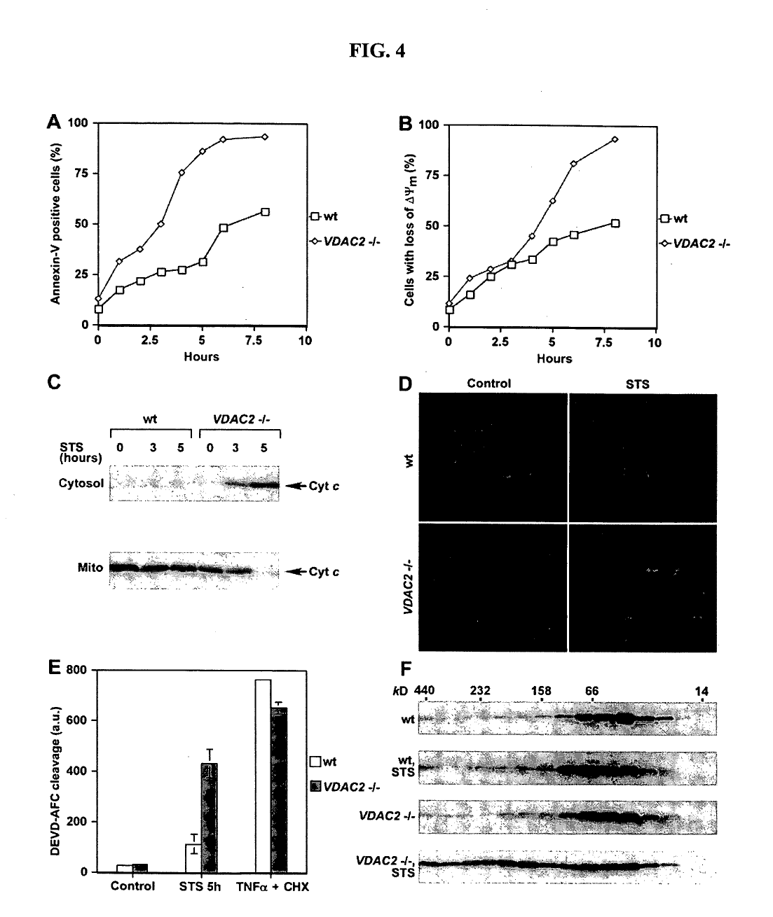

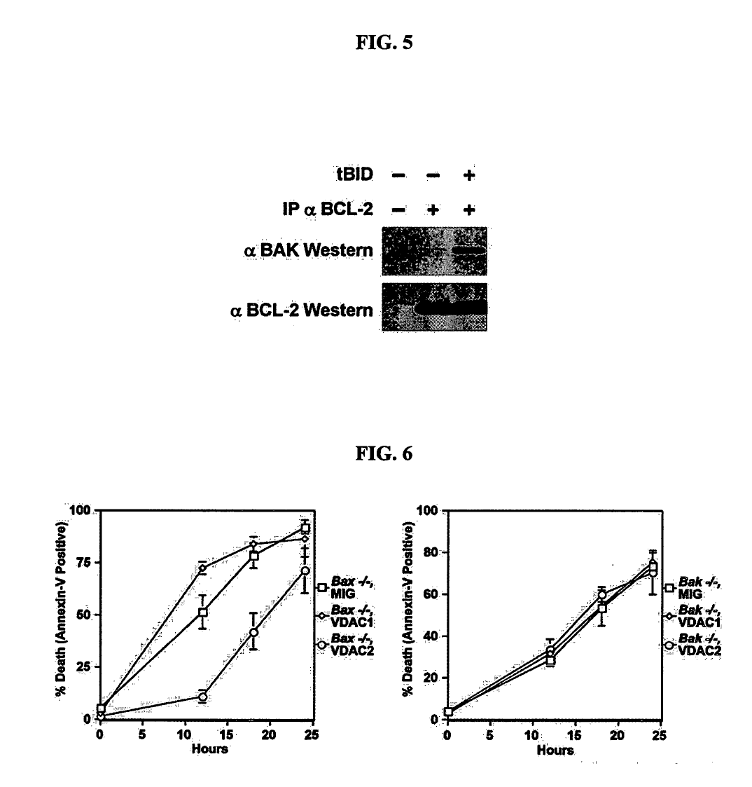

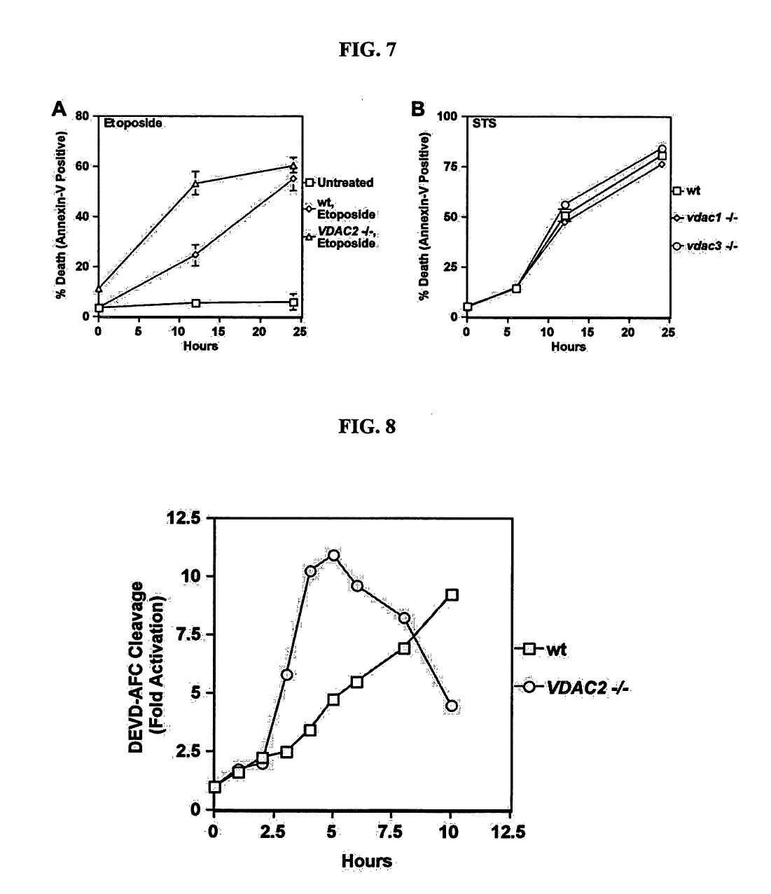

vdac2 -/- MEF cells responded to staurosporine with accelerated surface exposure of phosphatidylserine (FIG. 4A) and more cells lost mitochondrial transmembrane potential (ΔΨm) as measured by tetramethylrhodamine ethyl ester (TMRE) fluorescence intensity (FIG. 4B). vdac2 -/- cells treated with staurosporine exhibited partial release of cytochrome c into the cytosol by 3 hours and complete release by 5 hours, times at which wt cells had not initiated any release of cytochrome c (FIG. 4C). Immunohistochemistry on wt cells confirmed that cytochrome c remained in a punctate mitochondrial pattern at 4 hours after staurosporine treatment but was diffusely cytosolic in vdac2-deficient cells (FIG. 4D). vdac2 -/- cells demonstrated activity of effector caspases 3 & 7 earlier than did wt cells after staurosporine treatment (FIG. 4E and fig. S4). In contrast, the response of wt- and vdac2-dificient cells was similar when the extrinsic death pathway was activated by TNFα (FIG. 4E). These findings prompted us to examine vdac2-null cells for changes occurring before mitochondrial damage. BAK already underwent oligomerization within 5 hours after staurosporine treatment in vdac2 -/- cells but not wt cells, as assessed by gel filtration (FIG. 4F) (X. Roucou, S. Montessuit, B. Antonsson, J. C. Martinou, Biochem J 368, 915-21. (2002)). This conformational activation of BAK coincides with the timing of complete release of cytochrome c and peak caspase activity. In contrast, wt cells did not display BAK oligomerization until ˜10 hours after treatment. In the absence of VDAC2, the crosslinker BMH did not efficiently capture these higher order BAK multimers, suggesting their conformation is altered. Thus, in the absence of inhibition by VDAC2, BAK demonstrated an enhanced allosteric conformational activation resulting in the release of cytochrome c, caspase activation, and mitochondrial dysfunction responsible for an increased susceptibility to apoptotic death.

### Example 6

### Interactions of BH3-Only Molecules with VDAC2

As described above, the BH3-only subset of BCL-2 family members includes pro-apoptotic molecules that bear sequence homology to the remaining multi-domain family members, only with the minimal death domain, the BH3 amphipathic a helix. Following activation of BH3-only molecules, including tBID, BIM or PUMA, VDAC2 and BAK are dissociated, and BAK homo-oligomerizes to permeabilize the outer mitochondrial membrane. However, any potential interaction of a BH3-only molecule and the BAK molecule was found to be short-lived, as the homo-oligomerized BAK species does not contain a BH3-only molecule. The experiments described herein were used to assess whether BH3-only molecules demonstrate more stable interactions with VDAC2, as part of the death activation sequence.

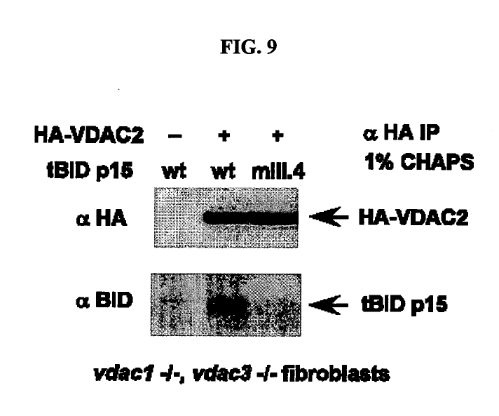

The activated form of BID, p15 tBID, was first examined in cells expressing only VDAC2 and not the other isoforms, VDAC1 or VDAC3. Mitochondria isolated from vdac1 -/- , vdac3 -/- mouse fibroblasts with or without retroviral transduction of N-terminal HA-tagged VDAC2 were incubated with in vitro transcribed, translated wild-type or mutant tBID (mIII.4, G94E) protein at 30° C. for 30 min, followed by anti-HA immunoprecipitation in 1% CHAPS buffer. The immunoprecipitates were analyzed by an immunoblot developed with anti-HA-Biotin or anti-BID as indicated in FIG. 9. As seen in lane 2 of FIG. 9, wild-type (wt) p15 tBID associated with VDAC2. Moreover, as seen in lane 3 of FIG. 9, the interaction between TBID and VDAC2 required an intact BH3 domain of tBID, as the mutant BH3 domain of TBID, the m111.4 mutant, did not recognize VDAC2. Thus, VDAC2 was found to interact with truncated BID (tBID) through the BH3 domain.

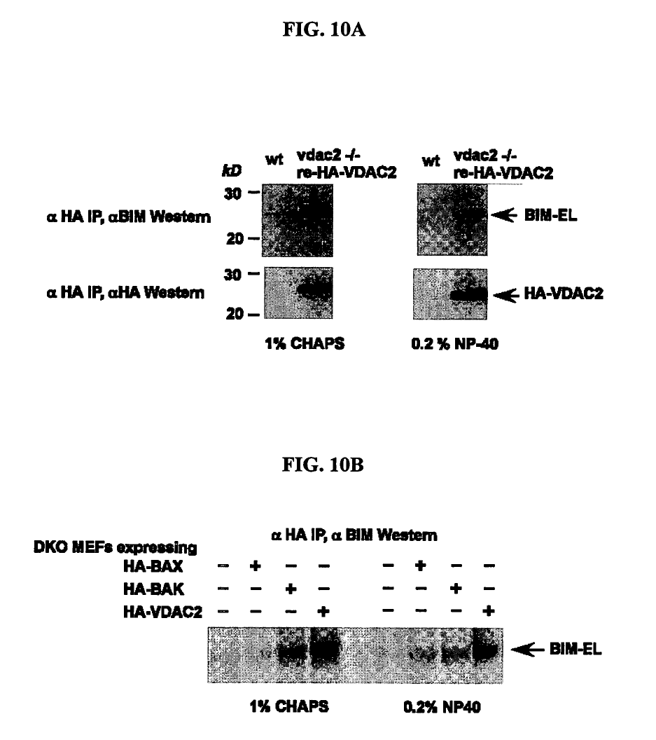

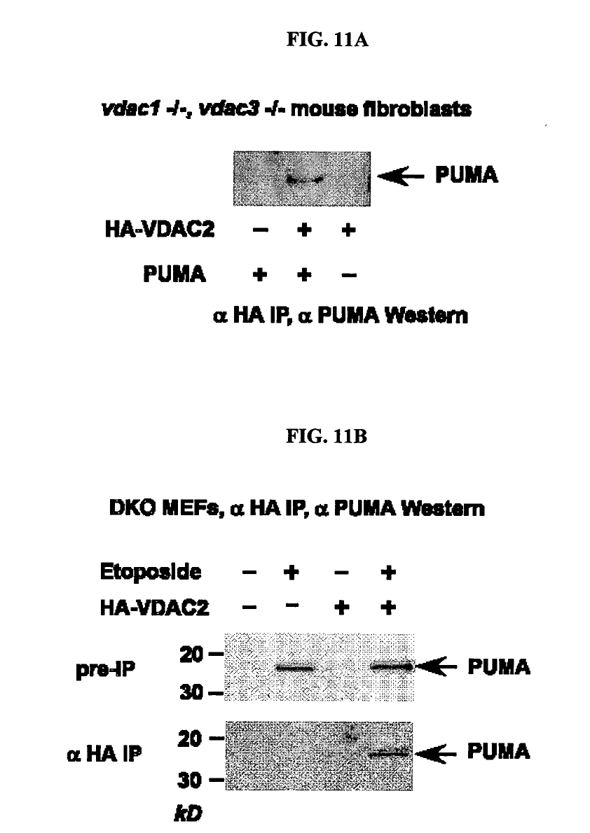

Similarly, the BH3-only molecule BIM-EL also displayed a strong interaction with VDAC-2 (FIG. 10A). Mitochondria isolated from wild-type MEFs or vdac2 -/- mouse embryonic fibroblasts (MEFS) with retroviral transduction of N-terminal HA-tagged VDAC2 were solubilized in either 1% CHAPS or 0.2% NP40 buffer, followed by anti-HA immunoprecipitation. The immunoprecipitates were analyzed by an immunoblot developed with either anti-BIM or anti-HA-Biotin as indicated in FIG. 10A.

The strength of association between BIM-EL and VDAC2 was compared to the strength of the association, if any, between BIM-EL and BAK or BAX. Mitochondria isolated from Bax, Bak doubly deficient MEFs transduced with retrovirus expressing N-terminal HA-tagged BAX, BAK, or VDAC2 were solubilized in 1% CHAPS or 0.2% NP-40 buffer, followed by anti-HA immunoprecipitation. The immunoprecipitates were analyzed by an immunoblot developed with anti-BIM. As seen in FIG. 10B, BIM-EL was shown to associate more strongly with VDAC2 than BAX or BAK, which indicates that the primary role of BIM-EL in activating BAK is a displacement reaction that removes VDAC2 from BAK. These findings indicate that BH3-only competition for VDAC2 frees BAK, which, in the absence of its specific inhibitor VDAC2, is prone to auto-activating homo-oligomerization.

The BH3 only molecule PUMA, which is activated by DNA damage, e.g., by etoposides, demonstrated a stable interaction with VDAC2 (FIGS. 11A and 11B). For FIG. 11A, mitochondria isolated from vdac1 -/-, vdac3 -/- mouse fibroblasts with or without retroviral transduction of N-terminal HA-tagged VDAC2 were incubated with in vitro transcribed, translated PUMA protein at 30° C. for 30 min, followed by anti-HA immunoprecipitation in 1% CHAPS buffer. The immunoprecipitates were analyzed by an immunoblot developed with anti-PUMA. For FIG. 11B, mitochondria were isolated from Bax, Bak doubly deficient MEFs with or without retroviral transduction of N-terminal HA-tagged VDAC2 before or after etoposide treatment (10 μM) for 20 hours, followed by anti-HA immunoprecipitation in 1% CHAPS buffer. The immunoprecipitates were analyzed by an immunoblot developed with anti-PUMA (FIG. 11B, lower panel). The upper panel of FIG. 11B demonstrates an anti-PUMA immunoblot before immunoprecipitation.

Thus, several BH3-only proteins and BH3 domains have been found to demonstrate specific interactions with VDAC2 accompanying the activation of BAK.

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for preventing apoptosis in a cell, said method comprising contacting said cell with a wild-type VDAC2 protein, aVDAC2 mimetic or a VDAC2/BAK stabilizer compound, wherein the wild-type VDAC2 protein, VDAC2 mimetic or VDAC2/BAK stabilizer compound modulates interaction between a VDAC2 protein and a BAK protein to form a VDAC2/BAK complex, thereby preventing apoptosis.

2. The method of claim 1, wherein the cell is contacted with a VDAC2 mimetic that comprises an anti-apoptotic mutated VDAC2 peptide that binds the BAK protein.

3. A method for preventing or alleviating a symptom of an apoptosis-associated disorder in a subject, said method comprising administering a wild-type VDAC2 protein, a VDAC2 mimetic or a VDAC2/BAK stabilizer compound, wherein the wild-type VDAC2 protein, VDAC2 mimetic or VDAC2/BAK stabilizer compound modulates interaction between a VDAC2 protein and a BAK protein to form a VDAC2/BAK complex.

4. The method of claim 3, wherein the cell is contacted with a VDAC2 mimetic that comprises an anti-apoptotic mutated VDAC2 peptide that binds the BAK protein.

5. The method of claim 3, wherein the apoptosis-associated disorder is selected from the group consisting of stroke, myocardial infarction, hypertension, septic shock, organ transplantation and a neurodegenerative disorder.

6. The method of claim 5, wherein the neurodegenerative disorder is selected from the group consisting of Parkinson's disease, amytrophic lateral sclerosis (ALS), Alzheimer's disease, Huntington's disease and immune deficiency.

7. A method for promoting apoptosis in a cell, said method comprising contacting said cell with a VDAC2/BAK inhibitor compound, wherein the VDAC2/BAK inhibitor compound prevents a VDAC2 protein and a BAK protein from interacting and forming a stable VDAC2/BAK complex, thereby promoting apoptosis.

8. The method of claim 7, wherein the VDAC2/BAK inhibitor compound is selected from the group consisting of a BID protein, a BIM protein, a BAD protein, a NOXA protein, a BH3 domain peptide, a BID mutein, a BIM mutein, a BAD mutein, a NOXA protein, and a BH3 domain mutein.

9. The method of claim 7, wherein the BH3 domain peptide is a BH3 only peptide.

10. The method of claim 9, wherein the BH3 only peptide is selected from the group consisting of tBID, BIM-EL and PUMA.

11. The method of claim 7, wherein the VDAC2/BAK inhibitor compound is selected from the group consisting of an anti-VDAC2 antibody, a pro-apoptotic VDAC2 mutein polypeptide and a pro-apoptotic BAK mutein polypeptide.

12. The method of claim 11, wherein the pro-apoptotic VDAC2 mutein polypeptide interacts with endogenous BAK but does not inhibit homo-oligomerization of BAK.

13. The method of claim 11, wherein the pro-apoptotic BAK mutein polypeptide interacts with endogenous VDAC2, thereby preventing endogenous VDAC2 from binding to endogenous BAK.

14. A method of preventing or alleviating a symptom of a cell-proliferative disorder, the method comprising administering a VDAC2/BAK inhibitor compound, wherein said VDAC2/BAK inhibitor compound modulates interaction between a VDAC2 protein and a BAK protein to prevent formation of a VDAC2/BAK complex, thereby promoting apoptosis in a sufficient amount to alleviate the symptom.

15. The method of claim 14, wherein the BH3 domain peptide is a BH3 only peptide.

16. The method of claim 15, wherein the BH3 only peptide is selected from the group consisting of tBID, BIM-EL and PUMA.

17. The method of claim 14, wherein the VDAC2/BAK inhibitor compound is selected from the group consisting of an anti-VDAC2 antibody, a pro-apoptotic VDAC2 mutein polypeptide and a pro-apoptotic BAK mutein polypeptide.

18. The method of claim 17, wherein the pro-apoptotic VDAC2 mutein polypeptide interacts with endogenous BAK but does not inhibit homo-oligomerization of BAK.

19. The method of claim 17, wherein the pro-apoptotic BAK mutein polypeptide interacts with endogenous VDAC2, thereby preventing endogenous VDAC2 from binding to endogenous BAK.

20. The method of claim 14, wherein the cell-proliferative disorder is selected from the group consisting of cancer, non-malignant neoplasias, DNA viruses, lymphoproliferative conditions, arthritis, inflammation and autoimmune disorders.

21. The method of claim 20, wherein the DNA virus is selected from Epstein Barr virus, African swine fever virus and adenovirus.

22. A method for screening for a VDAC2 mimetic or a VDAC2/BAK stabilizer compound, comprising the steps of:
(a) contacting a cell population with a candidate compound, wherein said cell population comprises at least one cell having a VDAC2 protein and a BAK protein; and
(b) determining the level of apoptosis of said cell population;
wherein a decrease in said level of apoptosis in the presence of the candidate compound compared to a normal control level of apoptosis in the absence of the said candidate compound indicates said candidate compound is a VDAC2 mimetic or a VDAC2/BAK stabilizer compound.

23. The method of claim 22, wherein said cell is contacted in vivo or in vitro.

24. A method for screening for a VDAC2/BAK inhibitor compound, comprising the steps of:
(a) contacting a cell population with a candidate compound, wherein said cell population comprises at least one cell having a VDAC2 protein and a BAK protein; and
(b) determining the level of apoptosis said cell population;
wherein an increase of said level of apoptosis in the presence of said candidate compound compared to a normal control level of apoptosis in the absence of said compound indicates that said candidate compound is a VDAC2/BAK inhibitor compound.

25. The method of claim 24, wherein said cell is contacted in vivo or in vitro.

26. A method of screening for an anti-apoptotic compound comprising the steps of:
(a) contacting a cell population with a candidate compound, wherein said cell population comprises at least one cell having a VDAC2 protein and a BAK protein; and
(b) determining the level of apoptosis of said cell population;
wherein a decrease in said level of apoptosis in the presence of the candidate compound compared to a normal control level of apoptosis in the absence of the said candidate compound indicates said candidate compound is an anti-apoptotic compound, further wherein the anti-apoptotic compound prevents the release of the BAK protein from a VDAC2/BAK complex, thereby inhibiting apoptosis.

27. The method of claim 26, wherein the anti-apoptotic compound is a BH3 only mutein or a small molecule compound.
